SEQUENCE LISTING (6 sequences)

SEQ ID NO: 1 - DNA, 21 bp, synthetic construct
  source 1..21: mol_type = other DNA; note = Forward primer for ITGB6
aattgccaacccttgcagtag

SEQ ID NO: 2 - DNA, 23 bp, synthetic construct
  source 1..23: mol_type = other DNA; note = Reverse Primer for ITGB6
aatgtgcttgaatccaaatgtag

SEQ ID NO: 3 - DNA, 25 bp, synthetic construct
  source 1..25: mol_type = other DNA; note = Forward Primer for ALG9
gaatgaccagaatctagaagagcca

SEQ ID NO: 4 - DNA, 25 bp, synthetic construct
  source 1..25: mol_type = other DNA; note = Reverse Primer for ALG9
tctcatggtgtccaaatccactaaa

SEQ ID NO: 5 - DNA, 24 bp, synthetic construct
  source 1..24: mol_type = other DNA; note = Forward Primer for GAPDH
ctttgtcaagctcatttcctggta

SEQ ID NO: 6 - DNA, 17 bp, synthetic construct
  source 1..17: mol_type = other DNA; note = Reverse Primer for GAPDH
ggccatgaggtccacca

(19) United States
(12) Patent Application Publication
(10) Pub. No.: US 2024/0374510 A1
(43) Pub. Date: Nov. 14, 2024

(54) COMPOSITIONS, USES AND METHODS FOR TREATMENT OF PERIODONTAL DISEASE

(71) Applicant: THE UNIVERSITY OF BRITISH COLUMBIA, Vancouver (CA)

(72) Inventors: Hannu LARJAVA, Vancouver (CA); Leeni KOIVISTO, Vancouver (CA); Alberto BALDELLI, Vancouver (CA); Jiarui BI, Vancouver (CA); Lari HÄKKINEN, Vancouver (CA); Anubhav PRATAP-SINGH, Vancouver (CA)

(21) Appl. No.: 18/611,484

(22) Filed: Mar. 20, 2024

Related U.S. Application Data
(60) Provisional application No. 63/453,259, filed on Mar. 20, 2023.

(51) Int. Cl. A61K 9/00; A61K 9/50; A61K 31/5377

(52) U.S. Cl. CPC A61K 9/0063; A61K 9/5031; A61K 9/5047; A61K 31/5377

(57) ABSTRACT

A sustained-release pharmaceutical composition for localized treatment of periodontal disease (PD) comprises a suspension of microparticles. The microparticles comprise an epidermal growth factor receptor inhibitor (EGFRI), such as gefitinib, with a stabilizing sugar encapsulated in a shell comprising a plurality of polymers. The suspension may be introduced into a periodontal pocket and the EGFRI may be slowly released over a period of 350 hours or more. Methods for treating PD include administering dose units of the suspension directly into periodontal pockets of a patient. If necessary, administration may be repeated on a schedule of approximately 3 months. In an example formulation the polymers included cellulose acetate butyrate, poly lactic-co-glycolic acid and ethyl cellulose.

### CROSS-REFERENCE TO RELATED APPLICATIONS

This application claims the benefit under 35 U.S.C. § 119 of U.S. application No. 63/453,259 filed 20 Mar. 2023 and entitled APPROACH FOR TREATMENT OF PERIODONTAL DISEASE which is hereby incorporated herein by reference for all purposes.

### SEQUENCE LISTING

This application contains a sequence listing, which has been filed electronically in XML format and is hereby incorporated by reference herein in its entirety. The XML file containing the sequence listing was created on 20 Mar. 2024, is named U0081301_Sequence-lisint.xml and is 7 kilobytes in size.

### FIELD

The present invention relates to treatment of periodontal disease (PD) with epidermal growth factor receptor inhibitors (EGFRIs). Embodiments of the invention include dosage forms, compositions and medications which include EGFRI for treatment of PD, uses of certain dosage forms, compositions and/or medications which include EGFRI for treatment of PD, and methods for treatment of PD that use EGFRI.

### BACKGROUND

Periodontal disease is a common oral inflammatory condition that affects about one half of the world's adult population, with 10-15% suffering from severe PD. PD contributes to systemic inflammation and thus to systemic inflammatory diseases, such as cardiovascular disease, diabetes, Alzheimer's disease, rheumatoid arthritis, and cancer.

In healthy people, the junctional epithelium (JE) connects the gingiva to the tooth enamel and participates in immune response against bacteria. In PD, JE moves away from the enamel. This results in formation of a periodontal pocket. Such pockets provide environments in which bacterial biofilms can thrive between the tooth and the pocket epithelium (PE). Progression of PD can eventually lead to progressive inflammatory destruction of the soft and hard periodontal tissues (osteolysis) and a loss of tooth attachment. Although microbial challenge is necessary for the initiation of PD, the host inflammatory response is the primary driving force for the ensuing periodontal tissue destruction (Sedghi et al., 2021).

Periodontal health is maintained by the interplay between two molecules, integrin αvβ6 and transforming growth factor-β1 (TGF-β1). Integrin αvβ6 is an exclusively epithelial tissue-restricted cell surface receptor which is highly expressed in healthy JE and is the key activator of latent TGF-β1. TGF-β1 is the main anti-inflammatory cytokine in the JE and sustains ITGB6 expression via signaling mediator Smad3.

In PD, αvβ6 integrin levels are significantly diminished (Ghannad et al., 2008; Haapasalmi et al., 1995). Bacterial biofilms can suppress β6 integrin mRNA and protein expression in cultured gingival epithelial cells (GECs) by attenuating TGF-β1 signaling, leading to an enhanced pro-inflammatory response (Bi et al., 2017). Furthermore, exposure to biofilm components induced the activation of epidermal growth factor receptor (EGFR) and EGFR-mediated inhibition of TGF-β1/Smad3 signaling and, consequently, ITGB6 suppression.

The important role of αvβ6 integrin in the maintenance of periodontal health is demonstrated in patients with mutations in the ITGB6 gene, which encodes the rate-limiting subunit of the αvβ6 integrin heterodimer. Such patients suffer from severe PD (Ansar et al., 2016). In addition, β6 integrin-null mice spontaneously develop PD and inflammation in other epithelial tissues (Bi et al., 2018; Ghannad et al., 2008; Huang et al., 1996). The progression of PD may, thus, be caused by an imbalance between TGF-β1 and EGFR signaling in the PE.

(Bi et al., 2020) proposed that EGFR inhibition may provide a target for clinical therapies to prevent inflammation and bone loss in PD. The application of chemical EGFRIs, either locally or systemically, significantly reduced bone loss and inflammation in a ligature-induced experimental mouse PD model (Bi et al., 2019; Bi et al., 2020).

There is a need for practical and effective tools for reducing the impact of PD such as suitable medicaments, dosage forms, treatment regimes, and treatment methods. The availability of such tools could contribute significantly to enhancing general health.

### SUMMARY

The present invention has a number of aspects. These include, without limitation: medicaments (pharmaceutical compositions) for treatment of PD and/or preventing or delaying onset of PD; uses of microparticles containing at least one EGFRI for treating PD, preventing or delaying onset of PD, and/or making medicaments for treatment of PD; dosage regimens for medicaments for treatment of PD; methods for making medicaments for treatment of PD; and methods for treatment of PD.

One example aspect of the invention provides a sustained-release pharmaceutical composition for the localized treatment of periodontal disease. The pharmaceutical composition comprises a suspension of microparticles. The microparticles of the suspension comprise: an epidermal growth factor receptor inhibitor (EGFRI), a stabilizing sugar, and a shell. The shell comprises a plurality of polymers. The EGFRI, and the stabilizing sugar are encapsulated in the polymer shell.

In some embodiments, the plurality of polymers comprises three polymers, a higher molecular weight polymer, an intermediate molecular weight polymer and a lower molecular weight polymer wherein the higher molecular weight polymer has a molecular weight that is greater than a molecular weight of the intermediate molecular weight polymer and the molecular weight of the intermediate molecular weight polymer is greater than a molecular weight of the low molecular weight polymer. In some embodiments, the plural polymers are soluble in ethanol and have low solubility in water. In some embodiments, the higher molecular weight polymer is poly lactic-co-glycolic acid (PLGA). In some embodiments, the intermediate molecular weight polymer is cellulose acetate butyrate (CAB). In some embodiments, the low molecular weight polymer is ethyl cellulose (EC).

In some embodiments, the EGFRI is gefitinib.

In some embodiments, a weight ratio of the CAB to the PLGA to the EC is about 59:9:24.

In some embodiments, a ratio of the combined mass of the plural polymers to the mass of the stabilizing sugar in the microparticles is at least 3:1.

In some embodiments, the stabilizing sugar comprises mannose.

In some embodiments, the stabilizing sugar comprises trehalose.

In some embodiments, a projected area equivalent diameter of the microparticles is 20 μm or less.

In some embodiments, the projected area equivalent diameter of the microparticles is in the range of about 3 μm to about 10 μm.

In some embodiments, the microparticles are 3% to 7% gefitinib by weight.

In some embodiments, when the pharmaceutical composition is mixed with a liquid selected from artificial saliva and cell culture medium, a release rate of the gefitinib from the microparticles is sufficiently slow that no more than 95% of the gefitinib has been released into the liquid in a period of 350 hours.

In some embodiments, the sustained-release pharmaceutical composition comprises a gel and the microparticles are suspended in the gel.

In some embodiments, the shell comprises an outermost layer, an intermediate layer inwardly adjacent to the outermost layer and an inner layer inwardly adjacent to the intermediate layer. In some embodiments, the outermost layer comprises PLGA, the intermediate layer comprises CAB and the inner layer comprises EC.

In some embodiments, the microparticles have a surface morphology that is folded and crumpled.

Another example aspect of the invention provides a dosage regime for treating periodontal disease (PD). The dosage regime consists of administering into a periodontal pocket of a patient suffering from PD a dose unit of a sustained release pharmaceutical composition as described herein wherein the EGFRI comprises gefitinib, and the dose unit consists essentially of an amount of the suspension such that the microparticles of the dose unit contain a total weight of gefitinib in the range of 26 ng to 1 μg.

Another example aspect of the invention provides a method of treating periodontal disease (PD), comprising: administering a dose unit of a sustained release pharmaceutical composition as described herein into a periodontal pocket of a subject suffering from PD, wherein the dose unit contains a total weight of gefitinib in the range of 26 ng to 1 μg. In some embodiments the method comprises repealing administration of the dose unit to the subject on an administration schedule of 10 to 15 weeks until symptoms of PD are relieved.

Another aspect of the invention provides pharmaceutical compositions having any new and inventive feature, combination of features, or sub-combination of features as described herein.

Another aspect of the invention provides methods having any new and inventive steps, acts, combination of steps and/or acts or sub-combination of steps and/or acts as described herein.

Another aspect of the invention comprises a dosage regimen for treatment of PD comprising any new and inventive feature, combination of features, or sub-combination of features as described herein.

Further aspects and example embodiments are illustrated in the accompanying drawings and/or described in the following description.

It is emphasized that the invention relates to all combinations of the above features, even if these are recited in different claims.

### BRIEF DESCRIPTION OF THE DRAWINGS

The accompanying drawings illustrate non-limiting example embodiments of the invention.

FIGS. 1A to 1G are schematic drawings that illustrate stages of a method for making microparticles comprising an encapsulated EGFRI according to an example embodiment.

FIG. 2 is a graph showing particle size (projected area equivalent diameter) as a function of the ratio of the mass of bulk material (e.g. a stabilizer such as a suitable sugar) to the mass of polymers for microparticles made by spray drying a solution including dissolved solids according to a set of example formulations.

FIG. 3 is a graph showing particle size (projected area equivalent diameter) as a function of the weight % of dissolved solids in a set of solutions for microparticles made by spray dying the solutions.

FIG. 3 is an example FTIR spectrum for gefitinib.

FIG. 5 is a calibration curve showing the area of a peak in the HPLC chromatogram of gefitinib as a function of concentration of gefitinib.

FIGS. 6A to 6C are graphs that show cumulative release of gefitinib as a function of time for microparticles according to various formulations.

FIGS. 6D and 6E are graphs that show cumulative release of gefitinib from a dialysis bag into artificial saliva and cell culture medium respectively as a function of time.

FIGS. 7A to 7D are bar charts showing the effect of different concentrations of gefitinib-containing microparticles of selected formulations and different concentrations of corresponding gefitinib-free control microparticles on the growth of cells in a cultured human gingival epithelial cell (GEC) line.

FIG. 7E is a graph showing the effect of different concentrations of gefitinib on the growth of the cultured GEC cells.

FIG. 8A is a bar chart showing relative EGFR activation for gefitinib-containing microparticles of selected formulations and corresponding controls. FIG. 8B is a set of representative Western blots that were used to assess EGFR activation for the cases shown in FIG. 8A.

FIG. 9 is a bar chart that shows the relative ability of gefitinib-containing microparticles of selected formulations and selected controls to cancel HB-EGF-induced attenuation of the TGF-β1-mediated upregulation of ITGB6.

### DETAILED DESCRIPTION

Throughout the following description, specific details are set forth in order to provide a more thorough understanding of the invention. However, the invention may be practiced without these particulars. In other instances, well known elements have not been shown or described in detail to avoid unnecessarily obscuring the invention. Accordingly, the specification and drawings are to be regarded in an illustrative, rather than a restrictive sense.

One aspect of the present technology provides a medicament or pharmaceutical composition for treatment of PD that, when introduced into a periodontal pocket, releases an EGFRI over an extended period (e.g. a period of at least 1 week and preferably at least two weeks or three weeks or more). In some embodiments the EGFRI comprises gefitinib. In some embodiments the EGFRI is selected from the group consisting of: gefitinib, afatinib, erlotinib, lapatinib, dacomitinib, canertinib and mixtures of two or more of those. These EGFRIs are currently in human clinical use or in clinical trials. These EGFRIs have been found to be capable of dose-dependently blocking ITGB6 downregulation by EGFR ligand heparin-binding EGF-like growth factor (HB-EGF) (Bi et al., 2020).

In the pharmaceutical composition the EGFRI may be supplied as part of a composition in which the EGFRI is encapsulated within microparticles. The microparticles slowly release the EGFRI when the microparticles are in the environment found in periodontal pockets.

The microparticles may, for example comprise one or more polymer that forms a shell that encapsulates the EGFRI. The one or more polymer may comprise plural polymers that have molecular weights in different ranges.

In some embodiments, the plural polymers have molecular weights that differ significantly from one another. For example, an average molecular weight of one of the polymers that has the highest average molecular weight may be greater than the average molecular weight of one of the polymers that has the lowest average molecular weight by a factor of at least 2¼ or a factor of at least 4. In some embodiments the microparticles comprise three different polymers wherein an average molecular weight of the intermediate molecular weight polymer is at least 1½ times or at least 2 times greater than the average molecular weight of the lowest average molecular weight polymer and the average molecular weight of the highest average molecular weight polymer is at least 1½ times greater or at least 2 times greater than the average molecular weight of the intermediate molecular weight polymer.

In some embodiments the one of the polymers having the lowest average molecular weight has an average molecular weight in the range of ______ Da to ______ Da. In some embodiments the one of the polymers having the highest average molecular weight has an average molecular weight in the range of about 400 Da to 700 Da.

In some embodiments, the one or more polymers comprises a mixture of three polymers. A highest molecular weight polymer may, for example, have an average molecular weight in the range of about 40 kDa (kilo Daltons) to about 150 kDa. An intermediate molecular weight polymer may, for example, have an average molecular weight in the range of about 6 kDa to about 25 kDa. A lowest molecular weight polymer may, for example, have an average molecular weight in the range of about 350 Da to about 2 kDa. The three polymers may, for example, respectively have molecular weights of about 66-107 kDa, 12 kDa and about 450 Da. For example, the three polymers may be poly lactic-co-glycolic acid (PLGA)′ cellulose acetate butyrate (CAB), and ethyl cellulose (EC).

In some embodiments, the one or more polymer comprises plural polymers that are arranged in layers in shells of the microparticles. The layers may enclose a core comprising the EGFRI. In some embodiments the layers are arranged around the core such that highest molecular weight polymers are primarily found in an outer layer of the microparticles while lower molecular weight polymers are primarily found inside the outer layer. For example, where the three polymers are PLGA, CAB, and EC, the PLGA may form an outermost layer, the EC may form an innermost layer and the CAB may form an intermediate layer between the innermost and outermost layers.

The polymers may be selected to have low solubility in water (e.g. to be sparingly soluble or slightly soluble or to have a solubility in water not exceeding about 10 g/L. Using polymers that have a very low solubility in water (e.g. solubility in water on the order of about 0.1 g/L or on the order of about 1 g/L) can help to slow the release of an EGFRI encapsulated in the microparticles and to extend the time over which release of the EGFRI continues. Under the warm (e.g., body temperature) moist conditions in a periodontal pocket the polymer shells of the microparticles are designed to slowly break down. As this occurs the encapsulated EGFRI is slowly released into the periodontal pocket over an extended time period (e.g., 300 to 350 hours or longer).

In some embodiments, the one or more polymers are polymers that have a solubility in a selected solvent (e.g. ethanol) that is much greater than a solubility of the polymer in water. In some embodiments the selected solvent has the property of dissolving the EGFRI being incorporated in the microparticles. Using polymers that are soluble in a suitable solvent such as ethanol facilitates forming the microparticles by spray drying as described elsewhere herein.

In some embodiments, surfaces of the microparticles have a folded and/or crumpled configuration. In some embodiments at least most of the microparticles have a largest dimension that does not exceed 15 μm or 12 μm or 9 μm. Sizes of the microparticles may be characterized by a projected area equivalent diameter (PAED), which is the diameter of a circle that has the same projected area as a face of one of the microparticles. In some embodiments, the 95^{th} percentile PAED for the microparticles is 20 μm or less. In some embodiments, the microparticles have sizes in the range of about 3 μm to about 10 μm.

In some embodiments, the microparticles include a bulk material (which may comprise a stabilizer such as a suitable sugar). The bulk material may, for example, comprise disaccharide sugars (e.g., sucrose, trehalose, maltose, lactose, etc.) and/or polyols. To create the microparticles by spray-drying the bulk material should be soluble in the solvent (e.g. ethanol) used for spray drying.

The bulk material may, together with EGFRI, form a core of the microparticle. The bulk material may consist of or include a material that acts to stabilize the EGFRI (a stabilizer). The stabilizer may, for example, comprise a sugar. Mannose and trehalose are examples of sugars that are soluble in ethanol and can stabilize an EGFRI such as gefitinib.

In some embodiments, a ratio of the weight of the stabilizer to the weight of the EGFRI in the microparticles is about 4:1 or greater. In some embodiments a ratio of the weight of the stabilizer to the combined weight of the polymers in the microparticles is at least 1:1. In some embodiments, the ratio of the weight of the stabilizer to the combined weight of the polymers in the microparticles is at least 1½:1 or at least 2:1.

In some embodiments, approximately 5% of the weight of the microparticles is provided by the EGFRI (e.g. gefitinib).

The medicament according to one example embodiment comprises microparticles containing gefitinib as an active ingredient together with mannose encapsulated in a polymer shell made up of CAB, PLGA, and EC.

In some embodiments, the microparticles are mixed into a volume of a suitable biocompatible carrier, for example a suitable gel. The gel may, for example comprise a gel based on pluronic acid such as 27% pluronic acid. The amount of EGFRI-containing microparticles in the gel may be selected so that a concentration of the EGFRI in the gel does not exceed a value in the range of 10 μg/ml-200 μg/ml.

In some embodiments, the medicament is provided in pre-filled syringes containing a suspension of the microparticles. The suspension may, for example comprise the microparticles suspended in a gel. Doses of the suspension may be dispensed into periodontal pockets of a patient for treating and/or preventing PD. For example, in some embodiments each dose of the suspension may comprise in the range of about 26 ng (70 pmol) to about 1 μg (2.3 nmol) of gefitinib, where the microparticles release the gefitinib slowly over a period of about three weeks. For example, in some embodiments, one treatment may comprise applying about 50 μl to about 100 μl of the suspension to a periodontal pocket.

Another aspect of the invention provides a use of a medicament according to any of the embodiments described above for treatment of PD. The treatment may be applied at any stage of PD including advanced stages and early stages. Where treatment is applied at earlier stages of PD the treatment may reverse or delay advancement of the PD. In some embodiments, the use is associated with a dosage regimen that calls for re-application of the medicament into periodontal pockets of the patient once every three months, as needed.

Another aspect of the invention provides a use of an EGFRI for treatment of PD wherein the EGFRI is present in microparticles that comprise the EGFRI encapsulated in polymer (e.g. as described herein). The microparticles, when introduced into a periodontal pocket, may release the EGFRI over an extended period. The microparticles may have any of the compositions, characteristics and/or features as described herein.

Another aspect of the invention provides a use of microparticles comprising an EGFRI encapsulated in a shell comprising a mixture of polymers in the manufacture of a medicament for treating and/or preventing PD. The microparticles may have a core-shell structure, with the mixture of polymers forming the shell and the EGFRI on its own or in combination with a pharmaceutically acceptable stabilizer forming the core.

Another aspect of the invention provides a method for making a medicament for treatment of PD. The method comprises dissolving an EGFRI and one or more polymers in a solvent and creating microparticles which comprise the EGFRI by spray-drying the solution.

In some embodiments, the solvent is ethanol. Ethanol is advantageous because ethanol can dissolve gefitinib as well as suitable polymers that are relatively insoluble (e.g. sparingly soluble or slightly soluble) in the fluids that are typically present in periodontal pockets (e.g. water, serum).

One aspect of the present technology provides a medicament for treatment of PD that, when introduced into a periodontal pocket, releases an EGFRI over an extended period (e.g. a period of at least 1 week and preferably at least two weeks or three weeks or more).

Another aspect of the invention provides methods for treatment of PD. The methods involve introducing an EGFRI into one or more periodontal pockets of a patient in a dosage form that provides slow release of the EGFRI over a period of at least one week. The introduction of the EGFRI may be repeated at intervals in the range of 2 weeks to 4 months. In some embodiments, the introduction of the EGFRI into the periodontal pocket is performed together with routine dental appointments scheduled about once every three months. In some embodiments the EGFRI is introduced into the one or more periodontal pockets of the patient in a dosage form as described herein.

Localized application of EGFRIs in periodontal pockets to treat PD as described herein (e.g. by restoring αvβ6 integrin levels and attenuating periodontal inflammation and bone loss by correcting the TGF-β1-EGFR imbalance) may significantly reduce or avoid the side effects that are known to occur when the same EGFRIs are administered systemically, such as in the treatment of epithelial cancers. For example, gefitinib, which is a low molecular weight inhibitor of the EGFR tyrosine kinase domain, is used as a treatment for several types of cancer involving EGFR-positive metastatic non-small cells (Kirby et al., 2006). The side effects of systemic gefitinib therapy include rash, diarrhea, nausea, acne, loss of appetite, hair loss, and sore throat (Birnbaum and Ready, 2005).

The following sections describe research activities and their results that demonstrate the viability of the medicaments, uses, dosage regimens and methods described herein.

### Materials and Methods

The experiments described below were performed using the materials listed in Table 1.

TABLE 1
MATERIALS

| MATERIAL | SOURCE | PART NO., NOTES |
|---|---|---|
| gefitinib | Toronto Research Chemicals, | |
| | Toronto, ON, Canada | |
| ethanol | Sigma Aldrich Canada, | 64-17-5 |
| | Oakville, ON, Canada | |
| cellulose | Sigma Aldrich | 9004-36-8, molecular |
| acetate buyrate | | weight ~12 kDa |
| Poly(D, | Sigma Aldrich | P1941, lactide:glycolide |
| L-lactide-co- | | (75:25), molecular |
| glycolide) | | weight 66,000-107,000, |
| ethyl cellulose | Sigma Aldrich | 200689 |
| D-mannose | Sigma Aldrich | M8574 |
| D-mannitol, | Sigma Aldrich | M4125 |
| D-(+)-trehalose | Sigma Aldrich, | T9449 |
| dihydrate | | |
| sodium chloride | Sigma Aldrich | S9888 |
| (NaCl) | | |

Experiments were conducted to optimize the encapsulation of gefitinib in spray dried particles for slow release in treating PD. In these experiments, gefitinib was diluted in ethanol with a combination of three different polymers, CAB, PLGA; and EC, together with a bulk material which was selected from the group consisting of: D-mannose, D-mannitol, D-(+)-trehalose dehydrate and sodium chloride. The relative amounts of these materials in different formulations that were tested are set out in Table 2. These ingredients were selected for solubility in ethanol. The polymers were selected to include polymers having a wide range of molecular weights.

The experiments explored: the effect of total weight percentage (wt %) of dissolved solids in the ethanol solutions to be spray dried (“Group I”); the effect of changing the ratio of polymer mass to sugar mass in the solutions (“Group II”); the effect of using combinations of two of the three polymers (leaving out one of the three polymers) (“Group III”); the effect of varying the ratio of the mass of the different polymers (“Group IV”), and the effect of using different sugars (“Group V”). In Table 2 the weight percentages (wt. %) are stated relative to the total weight of the ethanol solutions. The difference between 100% and the sum of the wt. % of the solutes for each formulation is the wt. % of ethanol in the corresponding solution.

In these experiments, the solutions had a gefitinib concentration of 5 μg/ml except that a gefitinib concentration of 30 μg/ml was used in the experiments described below for determining the release rate of gefitinib.

TABLE 2

| | | EC | CAB | PLGA | Mannose | Mannitol | Trehalose | NaCl |
|---|---|---|---|---|---|---|---|---|
| Grp. | Comment | wt. % | wt. % | wt. % | wt. % | wt. % | wt. % | wt. % |
| I | total wt. % solutes | | | | | | | |
| | 12.15 | 5.4 | 1.8 | 0.45 | 4.5 | | | |
| | 6.075 | 2.7 | 0.9 | 0.225 | 2.25 | | | |
| | 2.025 | 0.9 | 0.3 | 0.075 | 0.75 | | | |
| II | wt. ratio bulk material:polymers | | | | | | | |
| | 0.12 | 0.9 | 0.52 | 0.3 | 0.2 | | | |
| | 0.64 | 0.75 | 0.3 | 0.12 | 0.75 | | | |
| | 0.81 | 0.68 | 0.28 | 0.1 | 0.86 | | | |
| | 1.09 | 0.59 | 0.24 | 0.09 | 1 | | | |
| | 3.57 | 0.3 | 0.1 | 0.02 | 1.5 | | | |
| III | 2 Polymers | | | | | | | |
| | EC/CAB (*1) | 0.61 | 0.31 | | 1 | | | |
| | EC/PLGA | 0.71 | | 0.21 | 1 | | | |
| | CAB/PLGA | | 0.54 | 0.38 | 1 | | | |
| IV | Polymer weight order | | | | | | | |
| | CAB/EC/PLGA (*2) | 0.24 | 0.59 | 0.09 | 1 | | | |
| | PLGA/CAB/EC (*3) | 0.09 | 0.24 | 0.59 | 1 | | | |
| | EC/PLGA/CAB | 0.59 | 0.09 | 0.24 | | | | |
| V | Bulk material (Stabilizer) | | | | | | | |
| | Mannitol | 0.1 | 0.01 | 0.02 | | 2 | | |
| | Trehalose (*4) | 0.1 | 0.01 | 0.02 | | | 2 | |
| | NaCl | 0.1 | 0.01 | 0.02 | | | | 2 |
| | Mannose | 0.1 | 0.01 | 0.02 | 2 | | | |

Microparticles made from solutions having each of the formulations listed in Table 2 were studied for morphology and stability. Microparticles made from the formulations marked “(*1)” to “(*4)” were analyzed for storage stability, release rate of gefitinib and cytotoxicity.

### Techniques

Spray drying is a technique that may be used to generate small particles. Spray drying may be performed by dissolving solids in a solvent and spraying the resulting solution to form droplets in an atmosphere in which the solvent evaporates from the droplets. Evaporation of the solvent leaves behind particles formed from the solids.

There are relatively few previous studies of spray dried compositions which were intended to release an active material at a slow release rate. The previous studies often show maximum release rates over only a few hours (Bajaj et al., 2021; Davis and Illum, 1999; Desai and Park, 2005; Palmieri et al., 2001).

In experiments to develop spray dried particles that provide a slow release of an EGFRI (e.g. gefitinib) suitable for treating PD, the spray drying conditions selected were air temperature of 30° C., liquid solution feed rate of 3 L/min, and airflow of 5 L/min. Spray drying was performed using a Buchi model 290 spray drier. Once extracted from the collector, the resulting powder was stored in a vacuum chamber until used for each analysis.

In one set of experiments, three ethanol-soluble polymers, CAB, PLGA, and EC were used to provide a shell to encapsulate an ethanol soluble EGFRI (e.g. gefitinib). According to the theory of particle formation set out in (Baldelli et al., 2015) it is expected that these polymers form a three-layered shell on spray dried particles. The three polymers have a broad range of molecular weights. Molecular weight influences the race for the shell formation in an evaporating droplet (Baldelli et al., 2015). To increase the stability of gefitinib, a stabilizer (in this case one sugar) was included in the solution.

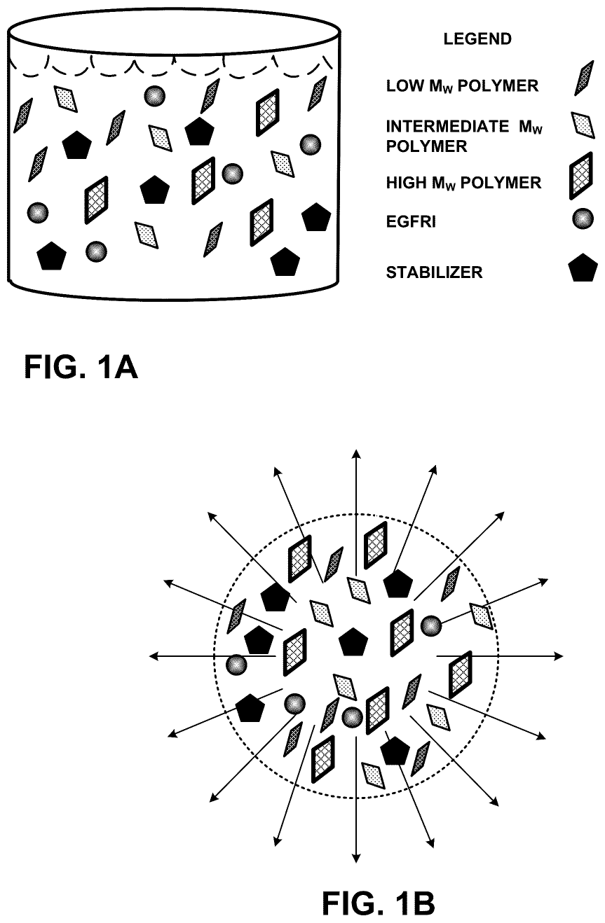

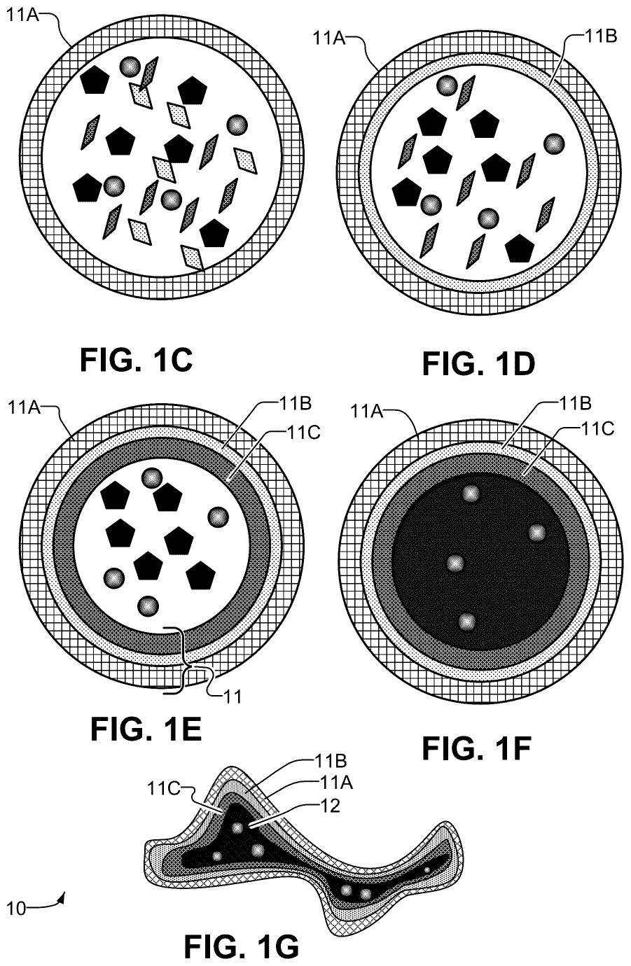

FIGS. 1A to 1G schematically illustrate stages in the formation of a spray-dried microparticle comprising a three-layered shell and containing gefitinib. In FIG. 1A three polymers having different molecular weights are dissolved in ethanol together with gefitinib and a stabilizing sugar. The resulting solution is sprayed to form droplets.

Particle formation during spray drying is driven by evaporation of a liquid component (e.g. ethanol). FIG. 1B schematically shows one such droplet. The radially directed arrows indicate evaporation of solvent (in this example ethanol). As the ethanol evaporates, the concentration of dissolved solids increases. When the concentration of a dissolved solid exceeds the solubility of the dissolved solid, the dissolved solid will start to form a precipitate. In an evaporating droplet, there can be significant gradients in the concentrations of different solutes. The diffusivity of the dissolved solids, together with their solubilities in the solvent and their initial concentrations can affect the order in which the solids precipitate and where in the droplet precipitation occurs

FIG. 1C illustrates formation of an outermost layer 11A of a shell 11 made up primarily of the one of the polymers that has the highest molecular weight. FIG. 1D illustrates formation of an intermediate layer 11B of shell 11. Intermediate layer 11B is formed inside outermost layer 11A and is made up primarily of the one of the polymers that has the second highest molecular weight. FIG. 1E illustrates formation of an innermost layer 11C of shell 11. Innermost layer 11C is formed inside intermediate layer 11B and is made up primarily of the one of the polymers that has the lowest molecular weight.

After innermost layer 11C has been formed, shell 11 surrounds remaining solution that contains gefitinib and the stabilizer. As shown in FIG. 1F, as the solvent continues to evaporate from the particle the stabilizing sugar precipitates inside shell 11. Eventually both the stabilizing sugar and gefitinib precipitate to form a solid core inside shell 11. As illustrated in FIG. 1G, continued evaporation of the solvent causes shell 11 to become folded and crumpled. After the particle is completely dried the microparticle has a solid core 12 containing gefitinib and the stabilizing sugar encapsulated in shell 11.

Study of microparticles formed as described herein showed that spray drying can be used to efficiently encapsulate gefitinib in microparticles and the resulting microparticles can release gefitinib slowly over periods on the order of 300 hours (12.5 days) or 350 hours or more.

An optimal formulation was made up of CAB, EC, PLGA, mannose and gefitinib (0.59, 0.24, 0.09, 1, and 0.005 mg/ml, respectively). Spray drying the optimal formulation resulted in creation of microparticles of 5.7±2.3 μm in diameter, encapsulation efficiency of 99.98%, and a slow release rate such that gefitinib was released over a period or more than 300 hours. A suspension of this microparticle formulation was found to block EGFR phosphorylation and to restore αvβ6 integrin levels in oral epithelial cells, while the respective control microparticles showed no effect.

### Characterization Techniques

### Morphology of Spray-Dried Microparticles

Powders made as described herein were transferred onto an SEM stub, on which a double side tape and a filter (0.2 μm pore size and 13 mm diameter, GSWP04700, EMD Millipore, Etobicoke, ON, Canada) were attached. The powders were then sputter coated with a 10-nm thick layer of gold using a sputter coater (Cressington 208 HR High-Resolution Sputter Coater, Cressington Scientific Instruments Ltd, Watford, UK).

A Helios NanoLab 650 Focused Ion Beam Scanning Electron Microscope (SEM; FEI Company, Hillsboro, OR, USA) was used to image the particles under the conditions of 13 mA and 5 kV. The imaging procedure is described in more detail in (Baldelli et al., 2020).

The projected area equivalent diameter (dₐ) was derived by measuring 300 particles per case. Image analysis was conducted using ImageJ software (https://imagej.nih.gov/ij/). More details on the image analysis procedure are described in (Baldelli and Vehring, 2016a; Trivanovic et al., 2019; Trivanovic et al., 2020).

### Stability of Gefitinib

The stability of gefitinib was verified by using two different characterization techniques: Fourier Transform InfraRed spectroscopy (FTIR) and High-Performance Liquid Chromatography (HPLC). The FTIR was used in the Attenuated Total Reflection mode. The IR spectrum can offer a qualitative representation for the understanding of the stability of an encapsulated drug. HPLC results can provide a more quantitative measure of the stability of gefitinib. The gefitinib quantification was conducted by using an Agilent 1100 series HPLC system (Agilent, Santa Clara, CA, USA) containing a quaternary pump, an autosampler, a column heater, and a DAD detector. Gefitinib was analyzed using a C18 column (Zorbax, 3.5 μm, 4.6 mm×150 mm; Agilent) at the wavelength of 280 nm. The mobile phase was composed of HPLC-grade acetonitrile and water in a gradient ratio from 10/90 to 100/0 for 10 min running. The mobile phase was pumped at a flow rate of 1.0 ml/min. The column temperature was set to 20° C.

### Storage Stability of Gefitinib in Spray-Dried Microparticles

The storage stability of gefitinib was tested using FTIR and HPLC using the protocols described above. Samples of powders containing spray-dried microparticles were stored at +4° C., and the stability of encapsulated gefitinib was tested two months from the date of production of the samples.

### Release Rate

The release rate of gefitinib from microparticles as described herein into each of two different liquids was tested. The two test liquids were: artificial saliva (SAE0149, Sigma Aldrich) and Dulbecco's Modified Eagle Medium (DMEM; Gibco, Life Technologies, Inc., Burlington, ON, Canada). These liquids were used to simulate the environment of the oral cavity and body fluid, respectively.

In each of these in-vitro experiments, a dry powder of spray-dried microparticles according to one of the tested formulations was mixed into the applicable test liquid at a concentration of 20 μg dry powder per ml of test liquid.

The in-vitro release behaviors of the formulations identified as (*1), (*2), (*3), and (*4) in Table 2 were each tested by the dialysis sack method (cutoff molecular weight 8 kDa; Spectra Por; Spectrum Chemical Mfg. Corp., New Brunswick, NJ, USA). All samples were incubated at room temperature with continuous shaking at 70 rpm. 5 mL of the fluid outside the dialysis sack (total volume of 20 mL) was withdrawn at the times of 4, 12, 24, 48, 120, 168, 336, and 502 hours. After each extraction the volume was immediately replenished with fresh dialysis fluid. The fluid was analyzed for gefitinib content by HPLC. The release rate of gefitinib from suspensions of dry powders in liquid was calculated from the ratio of released gefitinib to the total gefitinib encapsulated in the powders.

The release rate over time of unencapsulated gefitinib was also tested to verify that the dialysis bag did not significantly affect the accuracy of the release rates measured for the samples of spray-dried microparticles containing gefitinib. The release rate of unencapsulated gefitinib was measured according to the same protocol described above for the spray-dried microparticles.

### Effect on Cell Proliferation

The spontaneously immortalized human gingival epithelial cell line (GEC; (Mäkelä et al., 1998)) was maintained in a humidified incubator at +37° C. with 95% relative humidity and 5% CO₂ in DMEM supplemented with 23 mM sodium bicarbonate, 20 mM HEPES, 50 μg/ml streptomycin sulfate, 100 U/ml penicillin and 10% heat-inactivated fetal bovine serum (FBS; Gibco).

To compare the cytotoxicity profiles of the gefitinib-microparticles for formulations (*1) to (*4) to their respective control microparticles (no encapsulated gefitinib) and gefitinib alone, the microparticles were suspended at 1 mg/ml in FBS-free DMEM containing 0.1% tween 20 by repeated cycles of vortexing and centrifugation (12,000 rpm for 5 min). The remaining microparticle aggregates were then dispersed by low-power sonication with Branson Sonifier 250 (Branson Ultrasonics Corp., Danbury, CT, USA; five pulses at a power output of 1 and duty cycle of 10%). Gefitinib (10 mM) was dissolved in DMSO.

The MTT test (CellTiter Non-radioactive Cell Proliferation Assay, Promega, Madison, WI, USA) was used to assess the effect of the microparticle formulations on cell viability and proliferation (Twarużek et al., 2018). GECs were seeded into 96-well plate wells (15,000 cells per well; Costar, Corning, Inc., Corning, NY, USA) in their complete growth medium for 24 h. The cells were then washed once with phosphate-buffered saline (PBS) and further incubated in DMEM containing 2% FBS in the presence or absence of gefitinib- and control-microparticles (0.5-100 μg/ml of microparticles) or gefitinib (0.01-1 μM) for 24 h. The MTT tetrazolium dye was added for the final 3 h. The Solubilization/Stop Solution was then added to the culture wells for 24 h to solubilize the formazan product, and the absorbance at 570 nm was recorded with a microplate reader (iMark, Bio-Rad Laboratories, Hercules, CA, USA).

To determine the baseline cell levels at the beginning of the experiment, parallel cell wells were subjected to the MTT assay at the start of the test. At least four batches of spray-dried microparticles of each of formulations (*1) to (*4) were independently prepared. The testing was repeated at least ten times in separate experiments in duplicates.

### Efficacy of Epidermal Growth Factor Receptor Inhibition

GECs were seeded into cell culture dishes and allowed to grow to confluency for two days in their complete growth medium. The cells were then rinsed once with PBS and switched to FBS-free DMEM for 2 h in the presence or absence of: a suspension of gefitinib-microparticles of one of formulations (*1) to (*4) having 20 μg of the dry microparticles per ml of suspension; a suspension of control microparticles according to one of formulations (*1) to (*4) which did not include gefitinib; or 0.3 μM gefitinib.

HB-EGF (1 ng/ml; R&D Systems, Minneapolis, MN, USA) was then added to the cells for 10 min to stimulate EGFR activation. GECs were then washed once with PBS and lysed in 1× Laemmli sample buffer.

To assess the efficacy of the inhibition of EGFR activation by the microparticles, activation-dependent Tyr¹⁰⁶⁸ phosphorylation of EGFR relative to total EGFR protein expression was analyzed by Western blotting. Briefly, the cell lysates were separated by SDS/PAGE and transferred onto the Hybond Protran membrane (Amersham, Little Chalfont, Buckinghamshire, UK).

The membranes were incubated in Intercept Blocking Buffer (LI-COR Biosciences, Lincoln, NE, USA) for 1 h, followed by incubation with primary antibodies overnight at +4° C. The primary antibodies used were anti-pEGFR (Y1068; #3777) from Cell Signaling Technology (Danvers, MA, USA) and anti-EGFR (sc-373746) from Santa Cruz Biotechnology (Dallas, TX, USA). After incubation with species-appropriate IR dye-conjugated secondary antibodies (LI-COR) for 1 h, the blots were washed and scanned with LI-COR Odyssey Infrared Imaging system. The results were analyzed with Odyssey software (version 3.0).

### Rescue of β6 Integrin Gene Expression

GECs were seeded into cell culture dishes and allowed to grow to confluency for two days in their complete growth medium. The cells were then rinsed once with PBS and switched to FBS-free DMEM for 2 h in the presence or absence of one of: one of the suspensions of gefitinib-microparticles as above, one of the suspensions of respective control microparticles as above or 0.3 μM gefitinib. HB-EGF (5 ng/ml) and TGF-β1 (2 ng/ml; Millipore) were then added to the cells for a further 24 h.

The cells were then rinsed with PBS and used for RNA isolation and quantitative real-time reverse transcription PCR (RT-qPCR). Briefly, total RNA was extracted using PuroSPIN™ MINI Spin Columns (Luna Nanotech, Inc., Markham, ON, Canada) and the LogSpin method (Yaffe et al., 2012), and RNA concentration and purity were determined by spectrophotometry (NanoDrop One, Thermo Fisher Scientific, Waltham, MA, USA). 1 μg of total RNA was reverse-transcribed into cDNA using the High-Capacity cDNA Reverse Transcription Kit (Applied Biosystems, Thermo Fisher Scientific). The cDNA was diluted to a concentration with a threshold-cycle value well within the range of its standard curve. 5 μl of diluted cDNA was mixed with 10 μl of 2×iQ SYBR Green I Supermix (Bio-Rad) and 5 pmol of primers in each 96-well plate wells.

Asparagine-linked glycosylation 9 (ALG9) and glyceraldehyde-3-phosphate dehydrogenase (GAPDH) were used as reference genes for the target gene ITGB6 in triplicates. The PCR primers were synthesized by Integrated DNA Technologies (Coralville, IA, USA) and their sequences were: ITGB6: forward: 5′-AATTGCCAACCCTTGCAGTAG-3′ (SEQ ID NO: 1), reverse: 5′-AATGTGCTTGAATCCAAATGTAG-3′ (SEQ ID NO: 2); ALG9: forward: 5′-GAATGACCAGAATCTAGAAGAGCCA-3′ (SEQ ID NO: 3), reverse: 5′-TCTCATGGTGTCCAAATCCACTAAA-3′ (SEQ ID NO: 4); GAPDH: forward: 5′-CTTTGTCAAGCTCATTTCCTGGTA-3′ (SEQ ID NO: 5), reverse: 5′-GGCCATGAGGTCCACCA-3′ (SEQ ID NO: 6). Real-time PCR amplification was performed with the QuantStudio 3 Real-Time PCR System (Applied Biosystems) using a program of: 6 min at 95° C., followed by 40 cycles of 15 s at 94° C., 30 s at 60° C. and 20 s at 72° C. The data were analyzed using the Applied Biosystems Relative Quantification Analysis Module.

### Approach to Statistics

The approach used for determining statistics for the results of the tests described herein varied. For example, for determining the projected area equivalent diameter of microparticles, 300 of the microparticles were analyzed. Standard errors were determined by generating the standard deviations of the 300 measurements. In tests involving quantification of gefitinib by measuring the area of the characteristic peak of gefitinib in the HPLC chromatogram (e.g. when deriving the release rate of gefitinib), the area of this characteristic peak was measured three times. Standard errors were generated by the differences between these measurements.

All experiments involving cells were separately repeated at least five times. Multiple comparison tests were performed by one-way ANOVA, followed by post hoc comparison with the Tukey-Kramer Multiple Comparisons Test, using the Real Statistics Resource Pack software for Excel (Release 7.6; www.real-statistics.com). Statistical significance was set at p<0.05. Statistical significance for RT-qPCR data (performed in triplicates) was calculated using log 2-transformed data.

### Results

### Characterization of the Spray-Dried Microparticles

### Morphology

The morphological properties of spray-dried microparticles include dimension (dₐ) and shape.

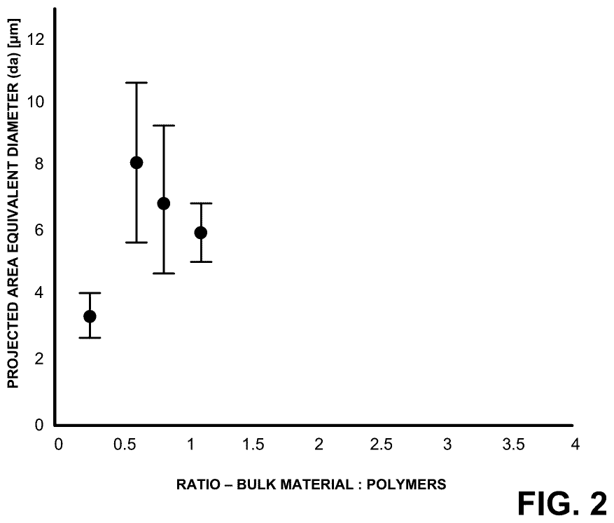

FIG. 2 is a graph that shows how particles size changes with the weight ratio of bulk material (sugar) to polymers in the solution that is spray dried to yield the microparticles.

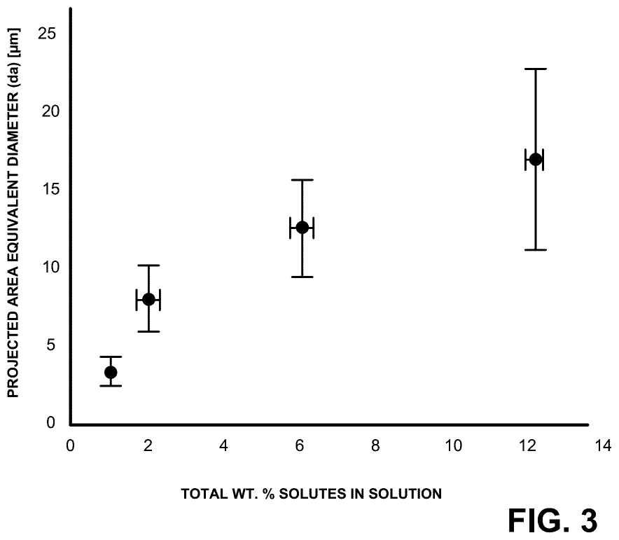

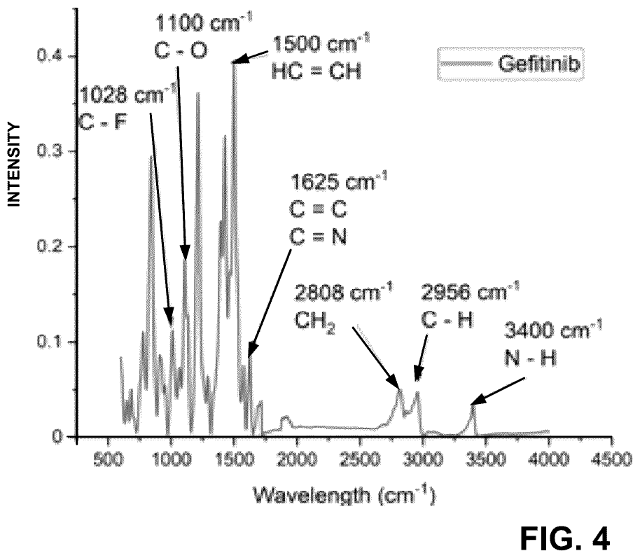

FIG. 3 is a graph that shows how particle size changes with total weight percentage of solids dissolved in the solution that is spray dried to yield the particles.

Table 3 shows sizes of spray-dried microparticles (expressed as projected area equivalent diameter) made using the different formulations listed in Table 2. Microparticle size, dₐ, increases sharply as the weight percentage of solids in the solution increases.

TABLE 3
PARTICLE SIZE (dₐ) FOR DIFFERENT
SPRAY DRIED FORMULATIONS

| | PARTICLE |
|---|---|
| FORMULATION | SIZE (dₐ) [μm] |
| EC/CAB (*1) | 7.1 ± 1.2 |
| EC/PLGA | 6.9 ± 0.8 |
| CAB/PLGA | 6.2 ± 1.8 |
| CAB/EC/PLGA (*2) | 6.3 ± 2.0 |
| PLGA/CAB/EC (*3) | 7.1 ± 1.4 |
| EC/PLGA/CAB | 6.7 ± 1.3 |
| Mannitol | 7.1 ± 1.2 |
| Trehalose (*4) | 5.9 ± 0.8 |
| NaCl | 5.3 ± 1.7 |
| Mannose | 6.3 ± 2.0 |

No trends were noted in the shapes of microparticles made using different formulations.

### Stability

The efficacy of encapsulation of gefitinib in microparticles as described herein was analyzed using FTIR and HPLC. The FTIR spectrum of gefitinib has a large number of peaks. This makes it difficult to obtain quantitative measurements of the amount of gefitinib present in different samples by comparison of FTIR spectra for the samples.

FIG. 4 is an FTIR spectrum of pure gefitinib. The FTIR spectrum includes several peaks corresponding to different vibrational modes of gefitinib that can be used to identify the presence of gefitinib. These peaks are labelled in FIG. 4.

The peaks labelled in FIG. 4 were qualitatively compared with corresponding peaks in FTIR spectra obtained for gefitinib in a solution with ethanol and for spray dried microparticles that encapsulated gefitinib and were made with the formulations listed in Table 2.

The gefitinib peaks in these FTIR spectra reasonably closely matched the FTIR spectrum of pure gefitinib (FIG. 4) except that the FTIR Spectra for: gefitinib in ethanol solution; microparticles for which the ratio of polymers/sugar were above 0.81, and microparticles of the formulations labeled EC/PGLA, Mannitol and Mannose. In these spectra the peaks attributable to gefitinib deviated from the pattern of FIG. 4.

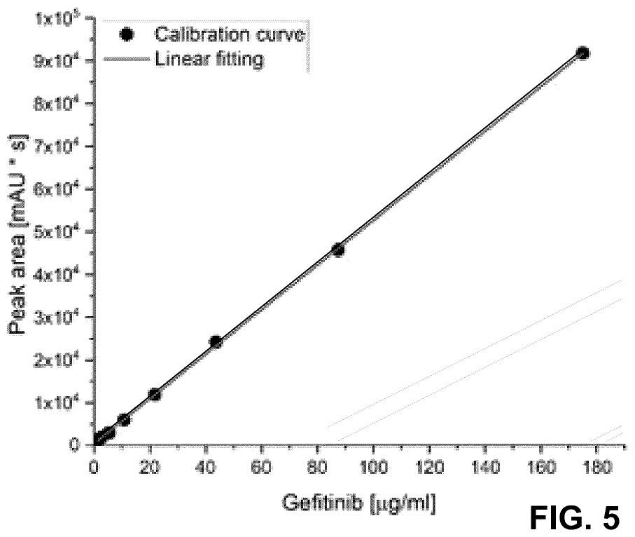

HPLC was used for quantitative comparisons of the amount of gefitinib in different samples. A main peak in the HPLC chromatogram of gefitinib (located at 13.02±0.06 minutes) was used for these comparisons. FIG. 5 shows a calibration curve that relates the area of this peak to the concentration of gefitinib in a sample that was used to make quantitative comparisons of the amount of gefitinib present in different samples. Table 4 shows results of these measurements.

TABLE 4
HPLC RESULTS

| | | Difference | | |
|---|---|---|---|---|
| | Comments/ | area | | η (2 |
| Group | Formulations | [mAU*s] | η | months |
| I | total wt. % solutes | | | |
| | 12.15 | −5351 | 61.83 | |
| | 6.075 | −1266 | 83.56 | |
| | 2.025 | −489 | 85.59 | |
| II | wt. ratio bulk | | | |
| | material: polymers | | | |
| | 0.12 | −3468 | 33.83 | |
| | 0.64 | −2089 | 79.15 | |
| | 0.81 | −2476 | 42.15 | |
| | 1.09 | −2509 | 33.97 | |
| | 3.57 | −390 | 97.80 | |
| III | 2 Polymers | | | |
| | EC/CAB (*1) | −77 | 99.89 | 78.45 |
| | EC/PLGA | −2488 | 89.66 | |
| | CAB/PLGA | −2508 | 21.74 | |
| IV | Polymer weight | | | |
| | order | | | |
| | CAB/EC/PLGA (*2) | −13.2 | 99.98 | 83.34 |
| | PLGA/CAB/EC (*3) | 10 | 99.95 | 87.23 |
| | EC/PLGA/CAB | −135 | 99.90 | |
| V | Bulk material | | | |
| | (Stabilizer) | | | |
| | Mannitol | −2446 | 90.58 | |
| | Trehalose (*4) | 49 | 99.98 | 90.34 |
| | NaCl | −5488 | 21.93 | |
| | Mannose | −464 | 88.88 | |

The value of η in Table 4 quantifies how much of the gefitinib contained in a solution remained in spray dried particles made using the solution. Ideally η=100%. In practice, η is less than 100% due to factors such as breakdown of gefitinib and/or losses in the spray drying process.

For all the formulations listed in Table 2, the stability of gefitinib was assessed based on the differences in the area of the chromatogram peak between encapsulated spray-dried microparticles and pure gefitinib, these calculated differences are provided in Table 4 in the column labeled “difference area [mAU*s]”.

The encapsulation efficiency, labeled as η in Table 4 indicates the quantity of gefitinib encapsulated during the process of spray drying as a percentage of the quantity of gefitinib in the solution input into the spray drying process. The last column shows the value of η for powders that have been stored at 4° C. for two months. The difference between the values of η and η (2 months) is a measure of stability of gefitinib in the spray-dried microparticles.

The formulations labeled (*1), (*2), (*3) and (*4) showed the smallest difference between the peak area of the pure and encapsulated gefitinib. These formulations also correspond to the highest encapsulation efficiencies. Therefore, these four formulations were selected for further studies on the release rate and cell proliferation.

### Release Rate

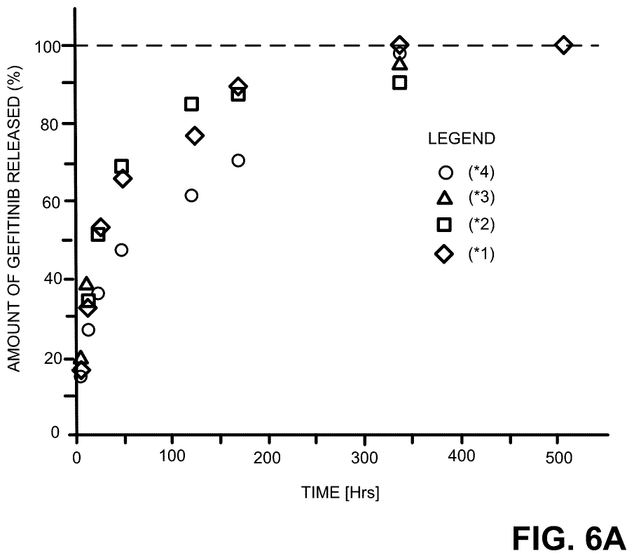

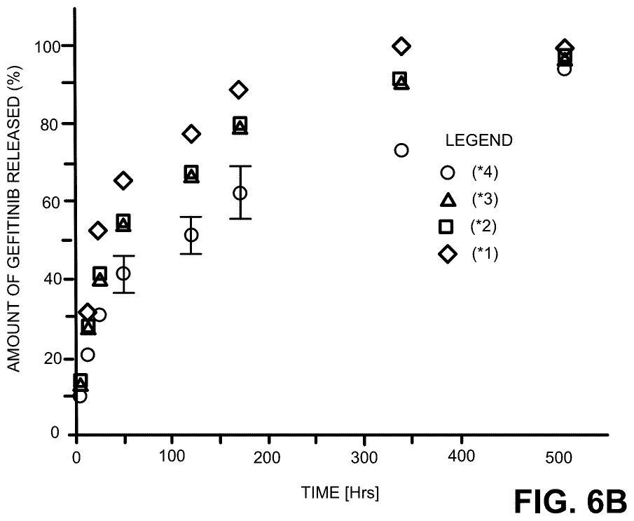

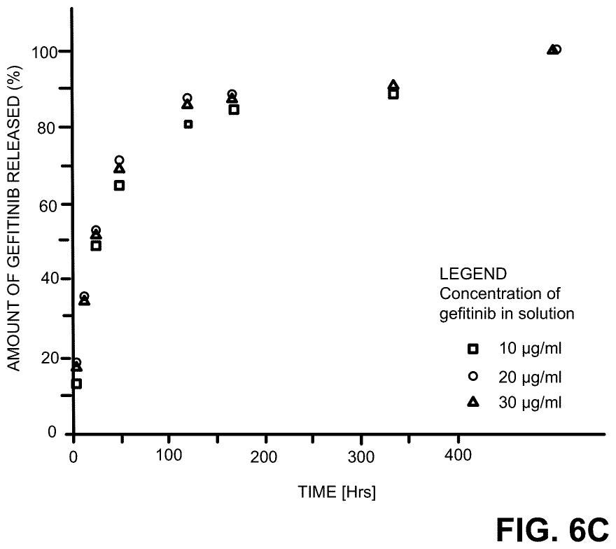

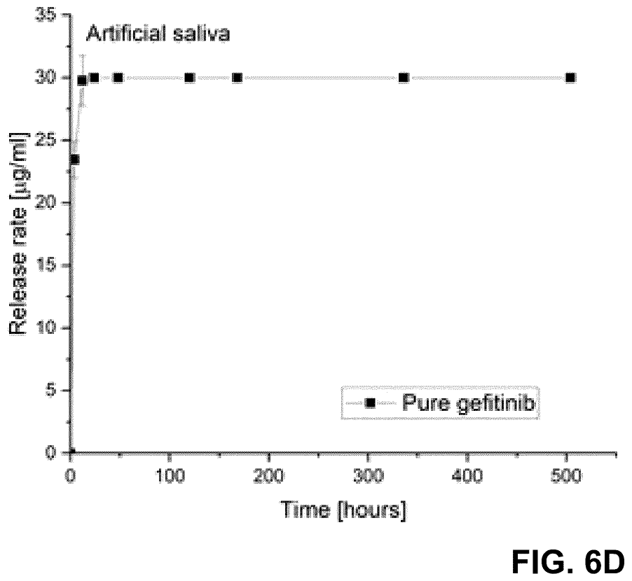

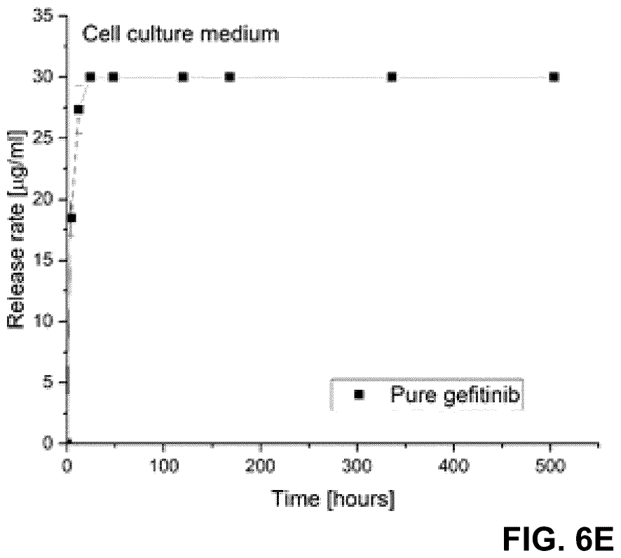

The release rate of gefitinib from microparticles of the formulations labeled (*1), (*2), (*3) and (*4) in Table 2 were tested in artificial saliva (FIG. 6A) and in Dulbecco's Modified Eagle Medium (FIG. 6B). The release rate was tested at different time steps with a maximum to three weeks. In FIGS. 6A and 6B, each point indicates the cumulative percentage of gefitinib released into the liquid at a time step measured relative to the time at which dry powders were introduced into the liquid (at time 0). The four tested formulations released gefitinib at similar rates. Each of the tested formulations had released all of the gefitinib originally contained in the spray dried microparticles after about a time of about three weeks. The formulation containing trehalose (*4) appeared to release gefitinib more slowly into saliva and cell culture liquid than the other tested formulations.

FIG. 6C shows release of gefitinib over time from microparticles of formulation (*2) for which the solution that was spray dried to yield the microparticles had different concentrations of gefitinib.

The release rate of unencapsulated gefitinib was measured using the same dialysis bag protocol and time steps as the encapsulated gefitinib. FIGS. 6D and 6E show release of pure unencapsulated gefitinib in artificial saliva and in cell culture medium respectively. FIGS. 6D and 6E show that the unencapsulated gefitinib is quickly released. Therefore, the dialysis sacks do not significantly delay the availability of released gefitinib in liquid outside of the dialysis sacks. The time scale (X-axis) of FIGS. 6D and 6E is the same as the time scale of FIGS. 6A and 6B. In FIGS. 6D and 6E the Y-axis indicates measured concentrations of gefitinib instead of the cumulative percentages indicated in FIGS. 6A and 6B.

### Characterization of the Biological Effectiveness of the Spray-Dried Microparticles

Biological functionality of the selected spray-dried gefitinib-containing microparticle preparations was tested in a set of cell-based assays designed to determine the specificity and effectiveness of the selected formulations for regulating cell proliferation, EGFR activation and β6 integrin gene expression.

### The Effect of the Spray-Dried Gefitinib Microparticles on Cell Growth

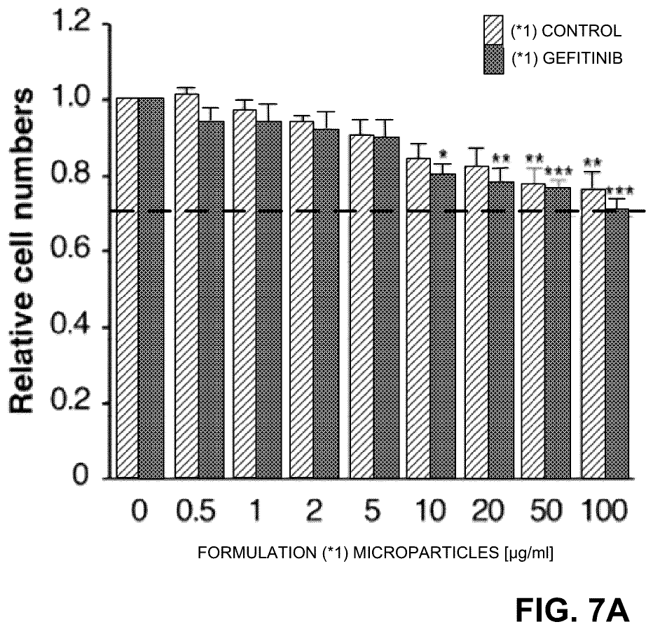

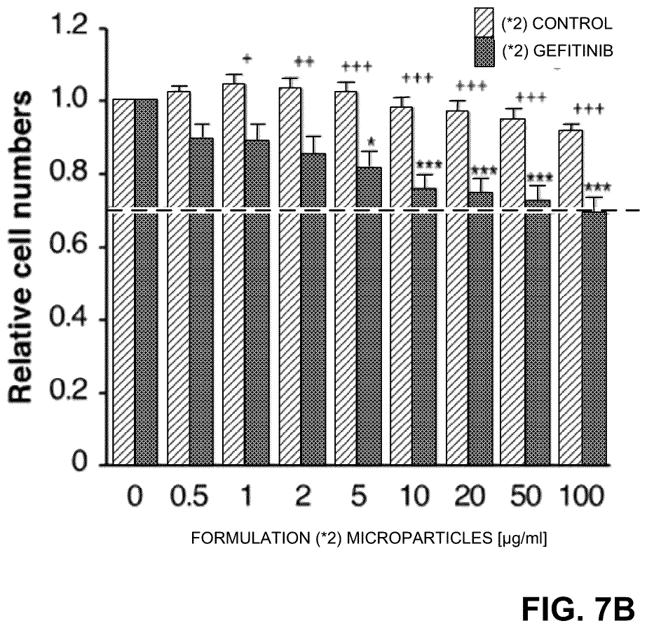

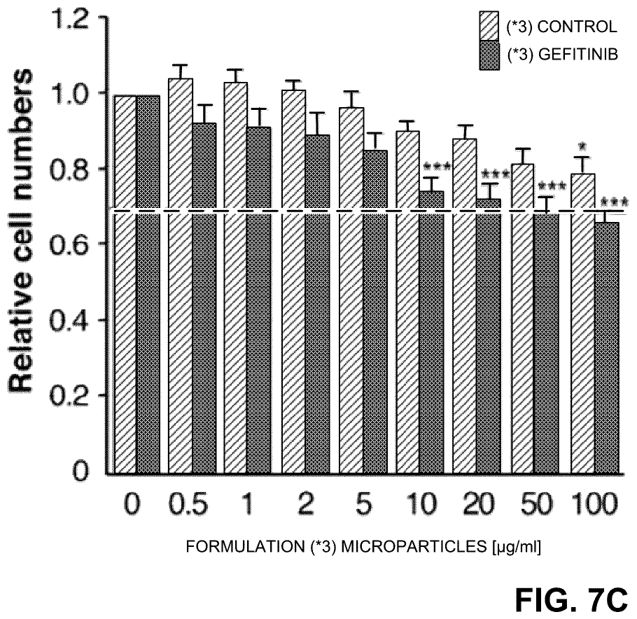

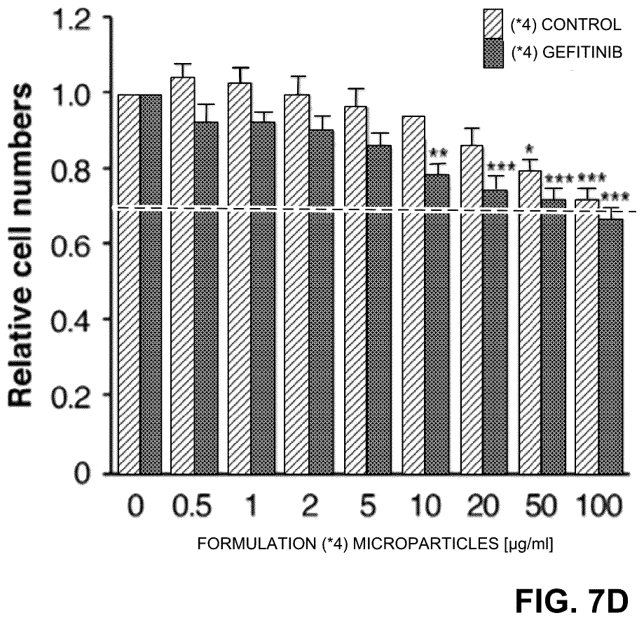

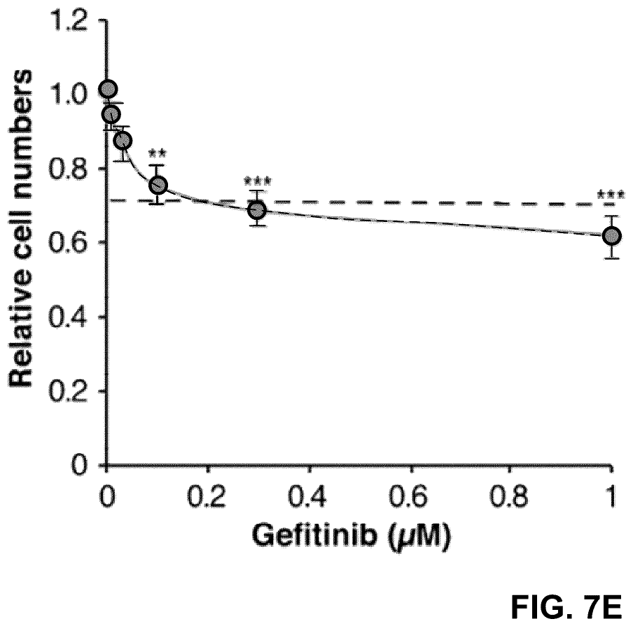

EGFR signaling sustains epithelial cell proliferation and survival (Wee and Wang, 2017). In order to assess the ability of the spray-dried gefitinib microparticles to inhibit cell proliferation and to assess the possible cytotoxicity of the microparticles, the gefitinib-containing microparticle formulations (*1) to (*4) (0-100 μg/ml) and corresponding gefitinib-free control microparticles were tested on the human gingival epithelial cell line described in (Bi et al., 2020). FIGS. 7A to 7D show the results of these tests. Unencapsulated gefitinib (0-1 μM) was also assessed for effectiveness at inhibiting cell proliferation. FIG. 7E shows the results of this test.

FIGS. 7A to 7D respectively show the effect of the spray-dried gefitinib-containing microparticles of formulations (*1), (*2), (*3) and (*4) and the corresponding control microparticles which lack gefitinib at concentrations in the range of 0-100 μg/ml on GEC cell growth. FIG. 7E shows the effect on GEC cell growth of unencapsulated gefitinib at concentrations of 0-1 μM.

In each test, the cells were exposed to the formulation being tested for 24 h and then analyzed for cell numbers using the MTT test. Absorbance values of the untreated samples in each replicate experiment were set to 1, and the mean±s.e.m. of the relative results of 10-18 replicated experiments is presented. *, relative to untreated cells; +, difference between the same concentration of gefitinib and control microparticles; */+, p<0.05; **/++, p<0.01; ***/+++, p<0.001. The horizontal dashed lines in FIGS. 7A to 7D correspond to the number of cells at the start of the experiment.

All four of the selected gefitinib-containing microparticle formulations were found to inhibit GEC growth. At concentrations of 20 μg/ml or more, GEC cell growth dropped to baseline levels (see FIGS. 7A to 7D). This corresponded to the GEC growth inhibition obtained with 0.3 μM gefitinib (see FIG. 7E).

Increasing the concentrations of the microparticles beyond 20 μg/ml did not result in a significant further drop in cell numbers.

For formulation (*2) the control microparticles were found to not affect GEC cell growth. For this formulation the cell growth inhibition of the gefitinib-containing microparticles exceeded that of the corresponding control microparticles by a statistically significant amount (see FIG. 7B).

For formulations (*1), (*3) and (*4), the gefitinib-free control microparticles had significant cell growth-reducing properties. This indicates a possible non-specific mechanism for these three formulations to regulate cell growth (See FIGS. 7A, 7C and 7D). As a result the differences in the cell numbers between the gefitinib-containing microparticles and the corresponding control microparticles were not statistically significant.

### The Effect of Spray-Dried Microparticles Containing Gefitinib on EGFR Activation

EGFR activation is associated with tyrosine-1068 phosphorylation of the receptor (Helin et al., 1991). The effect of gefitinib-containing microparticles according to formulations (*1) to (*4) (at 20 μg/ml) and the corresponding gefitinib-free control microparticles were tested for their ability to inhibit the phosphorylation of EGFR induced by its ligand HB-EGF by Western blotting. Gefitinib (0.3 μM) was used as a positive control for the inhibition.

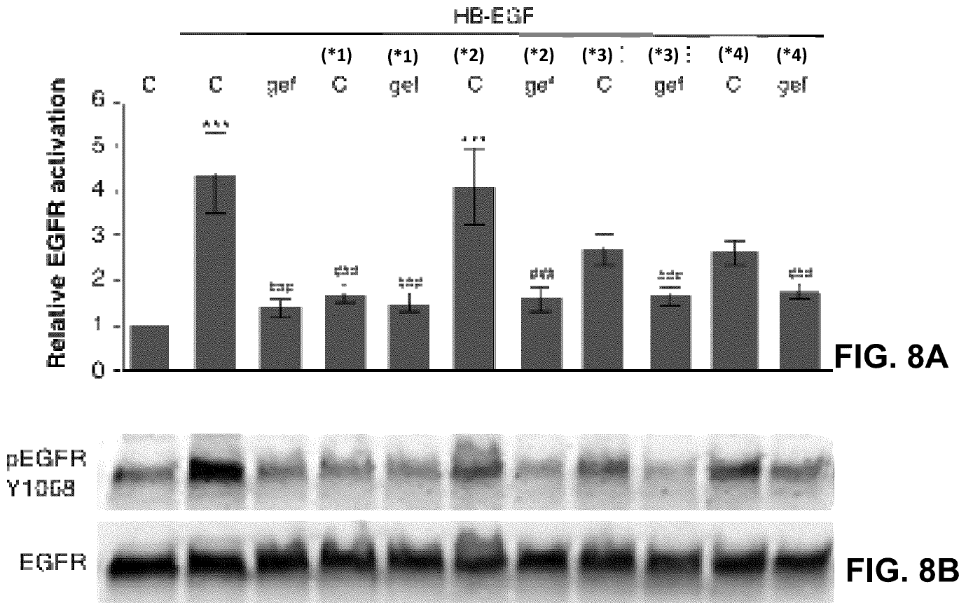

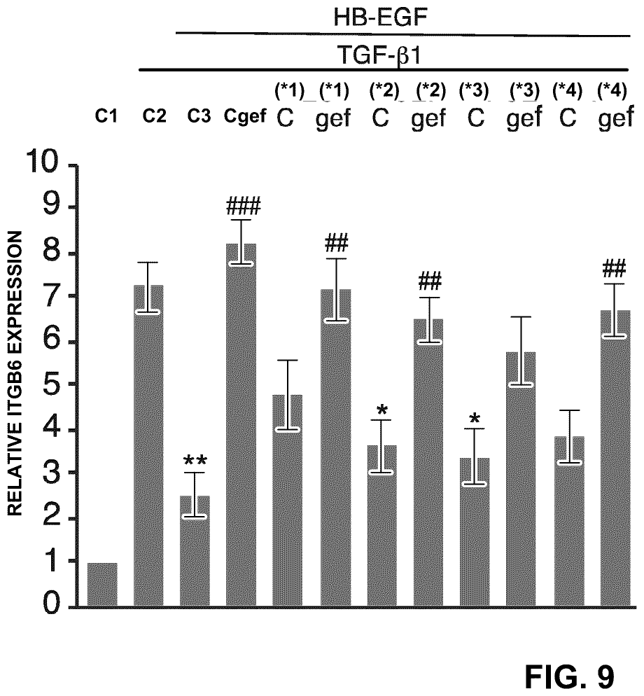

GECs were pre-treated with: gefitinib-containing microparticles according to each of formulations (*1) to (*4); and each of the corresponding gefitinib-fee controls (C), all at a dose of 20 μg/ml; and unencapsulated gefitinib at a dose of 0.3 μM. In each of these experiments, the GECs were left for 2 h after the pre-treatment and were then treated with HB-EGF (1 ng/ml) for 10 min. The GECs were then analyzed for EGFR phosphorylation relative to the total EGFR protein expression by Western blotting. The band intensity ratio for the untreated sample in each replicate experiment was set to 1. The mean±s.e.m. of the relative results of each of the nine replicate experiments are shown in FIG. 8A. In FIG. 8A, * indicates results that are relative to untreated cells; #indicates results that are relative to HB-EGF only-treated cells; and *** ###indicate p<0.001.

FIG. 8B shows the result of representative Western blots for each of the tested formulations and controls.

The results were consistent with the cell growth assays: while all gefitinib microparticle formulations completely and equally blocked the HB-EGF-induced EGFR phosphorylation, only formulation (*2) control microparticles had no inhibitory effect (see FIG. 8A). Control microparticles of formulations (*1), (*3) and (*4) were found to reduce EGFR phosphorylation even in the absence of gefitinib (see FIG. 8B). These results suggest that microparticles of formulations (*1), (*3) and (*4) may cause GEC growth inhibition by non-specific interference with EGFR activation.

### The Effect of Spray-Dried Gefitinib-Containing Microparticles on β6 Integrin Gene Expression

The cellular expression level of αvβ6 integrin is rate-limited at the level of β6 integrin mRNA expression (Xu et al., 2015). The balance of TGF-β1 and EGFR signaling determines ITGB6 expression in GECs, with EGFR ligands attenuating the upregulating effect of TGF-β1 (Bi et al., 2020). Gefitinib-containing microparticles of formulations (*1) to (*4) and their respective gefitinib-free control microparticles were tested to assess their ability to cancel HB-EGF-induced attenuation of the TGF-β1-mediated upregulation of ITGB6. Gefitinib was used as a positive control for the inhibition. TGF-β1 alone upregulated ITGB6 expression by about seven-fold, whereas the addition of HB-EGF reduced that upregulation by about 60% (See FIG. 9).

In FIG. 9: control C1 is for untreated cells; control C2 is for cells treated TGF-β1 only; control C1 is for cells treated both TGF-β1 and HB-EGF and control Cgef is for cells treated with TGF-β1, HB-EGF and gefitinib. Each of formulations (*1) to (*4) also includes a control C for treatment with gefitinib-free microparticles of the corresponding formulation and a bar gef for treatment with gefitinib-containing microparticles of the corresponding formulation.

GECs were pre-treated with gefitinib-containing microparticles of one of formulations (*1) to (*4) or the corresponding gefitinib-free microparticles (C) at a dose of 20 μg/ml or with unencapsulated gefitinib at a dose of 0.3 μM. Two hours after the pre-treatment the GECs were treated with TGF-β1 (2 ng/ml) with or without HB-EGF (5 ng/ml). 24 hours later the GECs were analyzed for their relative ITGB6 expression by RT-qPCR. The relative ITGB6 expression in the untreated cells in each replicate experiment was set to 1. The bars in FIG. 9 represent mean±s.e.m. of five replicate experiments. * indicates results relative to cells treated only with TGF-β1. #indicates results relative to cells treated with both TGF-β1 and HB-EGF. A single * indicates p<0.05; **/##represent p<0.01; ###, represents p<0.001.

In this assay, all four tested gefitinib-containing microparticle formulations were found to be effective at blocking the HB-EGF effect on ITGB6 expression. Control microparticles of formulation (*1) were also partially effective, whereas control microparticles of formulations (*3) and (*4) were found to be not effective. This suggests that the non-specific EGFR inhibitory effects of formulations (*3) and (*4) may occur by a mechanism that does not interfere with ITGB6 regulation.

### DISCUSSION

The experiments discussed above demonstrated encapsulation of gefitinib in spray-dried microparticles. The microparticles included a polymer shell that caused the encapsulated gefitinib to be slowly released when the microparticles were in contact with liquids similar to those found in periodontal pockets. The gefitinib released from the microparticles was shown to block EGFR and normalized ITGB6 levels in oral epithelial cells. The formulation labelled (*2) which included three polymers, namely CAB/EC/PLGA and mannose, was found to be the most effective among the tested formulations. Microparticles made from this formulation are believed to have three-layered shells.

In the present study, formation of the shell early in the particle formation process was ensured by using a highly volatile solvent (ethanol) and high molecular weight polymers, such as CAB, EC, and PLGA. These two conditions allowed the formation of at least one shell layer regardless of the total quantity of solids used, the ratio between polymers and sugar, the number of polymers used, and the ratios between two of the three polymers selected.

Shell formation is affected by the ratio between polymers and stabilizer (sugar). The highest encapsulation efficiency of gefitinib was reached when this ratio was above 3:1. This ratio also affects the morphology of microparticles without changing sizes (dₐ) of the microparticles. For low enough ratios of polymers to sugar, one can assume that the sugar, in this case, mannose, is distributed on the surface because if the sugar concentration is high enough and the polymer concentration is low enough then the sugar will precipitate first as the ethanol evaporates. The high viscosity of mannose can increase adhesion forces between microparticles, leading to the tendency of microparticles to form coagulates (Farías□Cervantes et al., 2017; Higgins and Mitchell, 2009). Agglomerates can impact the deposition location and the release rate of drugs delivered to different routes (Giuliani et al., 2018).

The crumpled and folded characteristics of spray-dried microparticles achieved by most of the formulations tends to be good for achieving a controlled release rate. Formulations as described herein that have greater quantities of dissolved solids tend to produce microparticles having surfaces that are more crumpled and folded upon spray drying (where the proportions of the components of the formulation are kept constant). As the total content of dissolved solids in the solution that is spray dried is increased, microparticle sizes tend to remain substantially unchanged but the encapsulation efficiency of gefitinib decreased.

Mannose and trehalose were shown to be effective stabilizers for gefitinib. Mannose advantageously has high solubility in ethanol. Formulations that included trehalose had the highest encapsulation efficiency. The storage stability of gefitinib was found to be very high when formulations were used that included trehalose as a stabilizer. Formulations that included mannose also demonstrated encapsulation efficiencies in excess of 99%.

The highest encapsulation efficiency tended to occur when formulations that included the highest molecular weight polymers were used. This could be due to earlier shell formation, offering higher protection for gefitinib.

The formulations that included three polymers were found to provide slow release of gefitinib over extended periods. For example, microparticles made by spray drying the formulations PLGA/CAB/EC and CAB/EC/PLGA, released gefitinib slowly enough that it took more than 350 hours to release 95% of the gefitinib in the microparticles. Microparticles made from formulations that included only two polymers tended to release gefitinib more rapidly than the three-polymer formulations. For example, microparticles made from the formulation EC/CAB were found to release more than 95% of the encapsulated gefitinib in less than 300 hours in both saliva and cell culture medium.

Of the formulations that released gefitinib slowly, only microparticles made from the formulation CAB/EC/PLGA appeared to specifically affect GEC cell growth, EGFR activation, and ITGB6 expression compared to the corresponding gefitinib-free control microparticles. This formulation is thought to produce microparticles that have three-layered shells with the outermost layer mainly made up of CAB, an intermediate layer mainly made up of ethyl-cellulose, and an inner shell mainly made up of PLGA. For all other tested three-polymer formulations, gefitinib-free control particles were observed to affect cell proliferation. The effect of the control microparticles on cell proliferation may be caused by butyrate (from CAB) which is known to decrease cell proliferation and to induce cell apoptosis in multiple types of cancers (Cao et al., 2019; Hong et al., 2015).

It is possible that control microparticles made from the formulation CAB/EC/PLGA had a smaller effect on cell proliferation than microparticles made from the other formulations because these microparticles had a thicker outer layer of CAB which may reduce the short term release of butyrate by being more impermeable than thinner CAB layers of other formulations. The same effect may explain the gradual release of gefitinib by microparticles made from formulation (*2) (CAB/EC/PLGA).

Microparticles comprising three-layered polymer shells were found to provide efficient encapsulation of gefitinib for slow release (e.g. over periods of about 2 weeks or more). Microparticles comprising three water non-soluble polymers, such as cellulose acetate butyrate, ethylcellulose, and poly lactic-co-glycolic acid, and a sugar, mannose, showed an encapsulation efficiency above 99%. The three layer shell provided a slow-release rate of the encapsulated gefitinib over an extended time. Furthermore, gefitinib-free control particles made from this formulation did not affect cell proliferation, thus showing minimal cytotoxicity. This formulation reduced EGFR activation and blocked EGFR-mediated ITGB6 downregulation.

### Interpretation of Terms

Where a component (e.g. a stabilizer, solvent, microparticle, etc.) is referred to herein, unless otherwise indicated, reference to that component (including a reference to a “means”) should be interpreted as including as equivalents of that component any component which performs the function of the described component (i.e., that is functionally equivalent), including components which are not structurally equivalent to the disclosed structure which performs the function in the illustrated exemplary embodiments of the invention.

Unless the context clearly requires otherwise, throughout the description and the claims:
“comprise”, “comprising”, and the like are to be construed in an inclusive sense, as opposed to an exclusive or exhaustive sense; that is to say, in the sense of “including, but not limited to”;
“connected”, “coupled”, or any variant thereof, means any connection or coupling, either direct or indirect, between two or more elements; the coupling or connection between the elements can be physical, logical, or a combination thereof;
“herein”, “above”, “below”, and words of similar import, when used to describe this specification, shall refer to this specification as a whole, and not to any particular portions of this specification;
“or”, in reference to a list of two or more items, covers all of the following interpretations of the word: any of the items in the list, all of the items in the list, and any combination of the items in the list;
the singular forms “a”, “an”, and “the” also include the meaning of any appropriate plural forms. These terms (“a”, “an”, and “the”) mean one or more unless stated otherwise;
“and/or” is used to indicate one or both stated cases may occur, for example A and/or B includes both (A and B) and (A or B);
“approximately” when applied to a numerical value means the numerical value±10%;
where a feature is described as being “optional” or “optionally” present or described as being present “in some embodiments” it is intended that the present disclosure encompasses embodiments where that feature is present and other embodiments where that feature is not necessarily present and other embodiments where that feature is excluded. Further, where any combination of features is described in this application this statement is intended to serve as antecedent basis for the use of exclusive terminology such as “solely,” “only” and the like in relation to the combination of features as well as the use of “negative” limitation(s)” to exclude the presence of other features; and
“first” and “second” are used for descriptive purposes and cannot be understood as indicating or implying relative importance or indicating the number of indicated technical features.

Words that indicate directions such as “vertical”, “transverse”, “horizontal”, “upward”, “downward”, “forward”, “backward”, “inward”, “outward”, “left”, “right”, “front”, “back”, “top”, “bottom”, “below”, “above”, “under”, and the like, used in this description and any accompanying claims (where present), depend on the specific orientation of the apparatus described and illustrated. The subject matter described herein may assume various alternative orientations. Accordingly, these directional terms are not strictly defined and should not be interpreted narrowly.

Where a range for a value is stated, the stated range includes all sub-ranges of the range. It is intended that the statement of a range supports the value being at an endpoint of the range as well as at any intervening value to the tenth of the unit of the lower limit of the range, as well as any subrange or sets of sub ranges of the range unless the context clearly dictates otherwise or any portion(s) of the stated range is specifically excluded. Where the stated range includes one or both endpoints of the range, ranges excluding either or both of those included endpoints are also included in the invention.

Certain numerical values described herein are preceded by “about”. In this context, “about” provides literal support for the exact numerical value that it precedes, the exact numerical value±5%, as well as all other numerical values that are near to or approximately equal to that numerical value. Unless otherwise indicated a particular numerical value is included in “about” a specifically recited numerical value where the particular numerical value provides the substantial equivalent of the specifically recited numerical value in the context in which the specifically recited numerical value is presented. For example, a statement that something has the numerical value of “about 10” is to be interpreted as: the set of statements:
in some embodiments the numerical value is 10;
in some embodiments the numerical value is in the range of 9.5 to 10.5;
   and if from the context the person of ordinary skill in the art would understand that values within a certain range are substantially equivalent to 10 because the values with the range would be understood to provide substantially the same result as the value 10 then “about 10” also includes:
in some embodiments the numerical value is in the range of C to D where C and D are respectively lower and upper endpoints of the range that encompasses all of those values that provide a substantial equivalent to the value 10.

Specific examples of systems, methods and apparatus have been described herein for purposes of illustration. These are only examples. The technology provided herein can be applied to systems other than the example systems described above. Many alterations, modifications, additions, omissions, and permutations are possible within the practice of this invention. This invention includes variations on described embodiments that would be apparent to the skilled addressee, including variations obtained by: replacing features, elements and/or acts with equivalent features, elements and/or acts; mixing and matching of features, elements and/or acts from different embodiments; combining features, elements and/or acts from embodiments as described herein with features, elements and/or acts of other technology; and/or omitting combining features, elements and/or acts from described embodiments.

As will be apparent to those of skill in the art upon reading this disclosure, each of the individual embodiments described and illustrated herein has discrete components and features which may be readily separated from or combined with the features of any other described embodiment(s) without departing from the scope of the present invention.

Any aspects described above in reference to apparatus may also apply to methods and vice versa.

Any recited method can be carried out in the order of events recited or in any other order which is logically possible. For example, while processes or blocks are presented in a given order, alternative examples may perform routines having steps, or employ systems having blocks, in a different order, and some processes or blocks may be deleted, moved, added, subdivided, combined, and/or modified to provide alternative or subcombinations. Each of these processes or blocks may be implemented in a variety of different ways. Also, while processes or blocks are at times shown as being performed in series, these processes or blocks may instead be performed in parallel, simultaneously or at different times.

Various features are described herein as being present in “some embodiments”. Such features are not mandatory and may not be present in all embodiments. Embodiments of the invention may include zero, any one or any combination of two or more of such features. All possible combinations of such features are contemplated by this disclosure even where such features are shown in different drawings and/or described in different sections or paragraphs. This is limited only to the extent that certain ones of such features are incompatible with other ones of such features in the sense that it would be impossible for a person of ordinary skill in the art to construct a practical embodiment that combines such incompatible features. Consequently, the description that “some embodiments” possess feature A and “some embodiments” possess feature B should be interpreted as an express indication that the inventors also contemplate embodiments which combine features A and B (unless the description states otherwise or features A and B are fundamentally incompatible). This is the case even if features A and B are illustrated in different drawings and/or mentioned in different paragraphs, sections or sentences.

It is therefore intended that the following appended claims and claims hereafter introduced are interpreted to include all such modifications, permutations, additions, omissions, and sub-combinations as may reasonably be inferred. The scope of the claims should not be limited by the preferred embodiments set forth in the examples, but should be given the broadest interpretation consistent with the description as a whole.

### REFERENCES

The following references provide background for the present technology.
1. Accardo, A., Morelli, G., 2015. Review peptide-targeted liposomes for selective drug delivery: Advantages and problematic issues. Peptide Science 104, 462-479.
2. Annes, J. P., Munger, J. S., Rifkin, D. B., 2003. Making sense of latent TGFbeta activation. J Cell Sci 116, 217-224.
3. Ansar, M., Jan, A., Santos-Cortez, R. L., Wang, X., Suliman, M., Acharya, A., Habib, R., Abbe, I., Ali, G., Lee, K., Smith, J. D., University of Washington Center for Mendelian, G., Nickerson, D. A., Shendure, J., Bamshad, M. J., Ahmad, W., Leal, S. M., 2016. Expansion of the spectrum of ITGB6-related disorders to adolescent alopecia, dentogingival abnormalities and intellectual disability. Eur J Hum Genet 24, 1223-1227.
4. Bajaj, S. R., Marathe, S. J., Singhal, R. S., 2021. Co-encapsulation of vitamins B12 and D3 using spray drying: Wall material optimization, product characterization, and release kinetics. Food Chemistry 335, 127642.
5. Baldelli, A., Boraey, M. A., Nobes, D. S., Vehring, R., 2015. Analysis of the particle formation process of structured microparticles. Molecular Pharmaceutics 12, 2562-2573.
6. Baldelli, A., Boraey, M. A., Oguzlu, H., Cidem, A., Rodriguez, A. P., Ong, H. X., Jiang, F., Bacca, M., Thamboo, A., Traini, D., 2022. Engineered nasal dry powder for the encapsulation of bioactive compounds. Drug Discovery Today, 27 (8), 2300-2308.
7. Baldelli, A., Power, R. M., Miles, R. E., Reid, J. P., Vehring, R., 2016. Effect of crystallization kinetics on the properties of spray dried microparticles. Aerosol Science and Technology 50, 693-704.
8. Baldelli, A., Trivanovic, U., Sipkens, T. A., Rogak, S. N., 2020. On determining soot maturity: A review of the role of microscopy- and spectroscopy-based techniques. Chemosphere, 126532.
9. Baldelli, A., Vehring, R., 2016a. Analysis of cohesion forces between monodisperse microparticles with rough surfaces. Colloids and Surfaces A: Physicochemical and Engineering Aspects 506, 179-189.
10. Baldelli, A., Vehring, R., 2016b. Control of the radial distribution of chemical components in spray-dried crystalline microparticles. Aerosol Science and Technology 50, 1130-1142.
11. Baldelli, A., Wells, S., Pratap-Singh, A., 2021. Impact of Product Formulation on Spray-Dried Microencapsulated Zinc for Food Fortification. Food and Bioprocess Technology 14, 2286-2301.
12. Bi, J., Dai, J., Koivisto, L., Larjava, M., Bi, L., Häkkinen, L., Larjava, H., 2019. Inflammasome and cytokine expression profiling in experimental periodontitis in the integrin β6 null mouse. Cytokine 114, 135-142.
13. Bi, J., Koivisto, L., Dai, J., Zhuang, D., Jiang, G., Larjava, M., Shen, Y., Bi, L., Liu, F., Haapasalo, M., 2020. Epidermal growth factor receptor signaling suppresses αvβ6 integrin and promotes periodontal inflammation and bone loss. Journal of Cell Science 133, jcs236588.
14. Bi, J., Koivisto, L., Pang, A., Li, M., Jiang, G., Aurora, S., Wang, Z., Owen, G. R., Dai, J., Shen, Y., 2017. Suppression of αvβ6 integrin expression by polymicrobial oral biofilms in gingival epithelial cells. Scientific Reports 7, 1-12.
15. Bian, W., Wang, M., Ahsan, B., Lin, S., Ren, Z., Huang, J.-A., Wang, J., 2018. Gefitinib-loaded nanoparticles with Folic acid-modified dextran surface prepared by flash nanoprecipitation. Chemistry Letters 47, 1405-1408.
16. Birnbaum, A., Ready, N., 2005. Gefitinib therapy for non-small cell lung cancer. Current Treatment Options in Oncology 6, 75-81.
17. Brunaugh, A. D., Wu, T., Kanapuram, S. R., Smyth, H. D., 2019. Effect of Particle Formation Process on Characteristics and Aerosol Performance of Respirable Protein Powders. Molecular Pharmaceutics 16, 4165-4180.
18. Buanz, A., Gurung, M., Gaisford, S., 2019. Crystallisation in printed droplets: understanding crystallisation of D-mannitol polymorphs. CrystEngComm 21, 2212-2219.
19. Cao, M., Zhang, Z., Han, S., Lu, X., 2019. Butyrate inhibits the proliferation and induces the apoptosis of colorectal cancer HCT116 cells via the deactivation of mTOR/S6K1 signaling mediated partly by SIRT1 downregulation. Molecular Medicine Reports 19, 3941-3947.
20. Chen, W., Palazzo, A., Hennink, W. E., Kok, R. J., 2017. Effect of particle size on drug loading and release kinetics of gefitinib-loaded PLGA microspheres. Molecular Pharmaceutics 14, 459-467.
21. Chitkara, D., Singh, S., Kumar, V., Danquah, M., Behrman, S. W., Kumar, N., Mahato, R. I., 2012. Micellar delivery of cyclopamine and gefitinib for treating pancreatic cancer. Molecular Pharmaceutics 9, 2350-2357.
22. Cullinan, M. P., Seymour, G. J., 2013. Periodontal disease and systemic illness: will the evidence ever be enough? Periodontol 2000 62, 271-286.
23. Davis, S., Illum, L., 1999. Sustained release chitosan microspheres prepared by novel spray drying methods. Journal of Microencapsulation 16, 343-355.
24. Desai, K. G. H., Park, H. J., 2005. Preparation and characterization of drug-loaded chitosan-tripolyphosphate microspheres by spray drying. Drug Development Research 64, 114-128.
25. Dominy, S. S., Lynch, C., Ermini, F., Benedyk, M., Marczyk, A., Konradi, A., Nguyen, M., Haditsch, U., Raha, D., Griffin, C., Holsinger, L. J., Arastu-Kapur, S., Kaba, S., Lee, A., Ryder, M. I., Potempa, B., Mydel, P., Hellvard, A., Adamowicz, K., Hasturk, H., Walker, G. D., Reynolds, E. C., Faull, R. L. M., Curtis, M. A., Dragunow, M., Potempa, J., 2019. Porphyromonas gingivalis in Alzheimer's disease brains: Evidence for disease causation and treatment with small-molecule inhibitors. Science Advances 5, eaau3333.
26. Drusch, S., Serfert, Y., Van Den Heuvel, A., Schwarz, K., 2006. Physicochemical characterization and oxidative stability of fish oil encapsulated in an amorphous matrix containing trehalose. Food Research International 39, 807-815.
27. Eke, P. I., Dye, B. A., Wei, L., Slade, G. D., Thornton-Evans, G. O., Borgnakke, W. S., Taylor, G. W., Page, R. C., Beck, J. D., Genco, R. J., 2015. Update on Prevalence of Periodontitis in Adults in the United States: NHANES 2009 to 2012. J Periodontol 86, 611-622.
28. Farías Cervantes, V. S., Chávez Rodríguez, A., Delgado Licon, E., Aguilar, J., Medrano Roldan, H., Andrade González, I., 2017. Effect of spray drying of agave fructans, nopal mucilage and aloe vera juice. Journal of Food Processing and Preservation 41, e13027.
29. Ferraz-Albani, L. A., Baldelli, A., Knapp, C. J., Jäger, W., Vehring, R., Nobes, D. S., Olfert, J. S., Kostiuk, L. W., 2017. Enhanced evaporation of microscale droplets with an infrared laser. Journal of Heat Transfer 139.
30. Ghannad, F., Nica, D., Fulle, M. I., Grenier, D., Putnins, E. E., Johnston, S., Eslami, A., Koivisto, L., Jiang, G., McKee, M. D., Hakkinen, L., Larjava, H., 2008. Absence of alphavbeta6 integrin is linked to initiation and progression of periodontal disease. Am J Pathol 172, 1271-1286.
31. Giuliani, A., Balducci, A. G., Zironi, E., Colombo, G., Bortolotti, F., Lorenzini, L., Galligioni, V., Pagliuca, G., Scagliarini, A., Calzà, L., 2018. In vivo nose-to-brain delivery of the hydrophilic antiviral ribavirin by microparticle agglomerates. Drug Delivery 25, 376-387.
32. Gradon, L., Sosnowski, T. R., 2014. Formation of particles for dry powder inhalers. Advanced Powder Technology 25, 43-55.
33. Guggina, L. M., Choi, A. W., Choi, J. N., 2017. EGFR inhibitors and cutaneous complications: a practical approach to management. Oncology and Therapy 5, 135-148.
34. Guo, Y., Baldelli, A., Singh, A., Fathordoobady, F., Kitts, D., Pratap-Singh, A., 2022. Production of high loading insulin nanoparticles suitable for oral delivery by spray drying and freeze drying techniques. Scientific Reports 12, 1-11.
35. Haapasalmi, K., Makela, M., Oksala, O., Heino, J., Yamada, K. M., Uitto, V. J., Larjava, H., 1995. Expression of epithelial adhesion proteins and integrins in chronic inflammation. Am J Pathol 147, 193-206.
36. Haque, M. A., Adhikari, B., 2015. Drying and denaturation of proteins in spray drying process. Handbook of Industrial Drying, 971-985.
37. Helin, K., Velu, T., Martin, P., Vass, W., Allevato, G., Lowy, D., Beguinot, L., 1991. The biological activity of the human epidermal growth factor receptor is positively regulated by its C-terminal tyrosines. Oncogene 6, 825-832.
38. Higgins, R., Mitchell, D., 2009. Mannose-binding lectin; the sugary-sticky side of transplantation. Transplantation 88, 149-150.
39. Hong, M. Y., Turner, N. D., Murphy, M. E., Carroll, R. J., Chapkin, R. S., Lupton, J. R., 2015. In Vivo regulation of colonic cell proliferation, differentiation, apoptosis, and P27Kip1 by dietary fish oil and butyrate in ratsfish oil and butyrate effects on colon cell kinetics. Cancer Prevention Research 8, 1076-1083.
40. Hu, Y., Zhang, J., Hu, H., Xu, S., Xu, L., Chen, E., 2020. Gefitinib encapsulation based on nano-liposomes for enhancing the curative effect of lung cancer. Cell Cycle 19, 3581-3594.
41. Huang, X. Z., Wu, J. F., Cass, D., Erle, D. J., Corry, D., Young, S. G., Farese, R. V., Jr., Sheppard, D., 1996. Inactivation of the integrin beta 6 subunit gene reveals a role of epithelial integrins in regulating inflammation in the lung and skin. J Cell Biol 133, 921-928.
42. Huntington, D. H., 2004. The influence of the spray drying process on product properties. Drying Technology 22, 1261-1287.
43. Kinane, D. F., Stathopoulou, P. G., Papapanou, P. N., 2017. Periodontal diseases. Nature Reviews Disease Primers 3, 1-14.
44. Kirby, A., A'hern, R., D'ambrosio, C., Tanay, M., Syrigos, K., Rogers, S., Box, C., Eccles, S., Nutting, C., Harrington, K., 2006. Gefitinib (ZD1839, Iressa™) as palliative treatment in recurrent or metastatic head and neck cancer. British Journal of Cancer 94, 631-636.
45. Kuruppu, A. I., Zhang, L., Collins, H., Turyanska, L., Thomas, N. R., Bradshaw, T. D., 2015. An apoferritin-based drug delivery system for the tyrosine kinase inhibitor gefitinib. Advanced Healthcare Materials 4, 2816-2821.
46. Larjava, H., Koivisto, L., Häkkinen, L., Heino, J., 2011. Epithelial integrins with special reference to oral epithelia. Journal of Dental Research 90, 1367-1376.
47. Li, Q., Cao, L., Tian, Y., Zhang, P., Ding, C., Lu, W., Jia, C., Shao, C., Liu, W., Wang, D., 2018. Butyrate suppresses the proliferation of colorectal cancer cells via targeting pyruvate kinase M2 and metabolic reprogramming. Molecular & Cellular Proteomics 17, 1531-1545.
48. Li, X., Wang, J., Li, S., Liu, Z., Zheng, Z., Zhang, Y., 2019. Development and evaluation of multifunctional poly (lactic-co-glycolic acid) nanoparticles embedded in carboxymethyl β-glucan porous microcapsules as a novel drug delivery system for gefitinib. Pharmaceutics 11, 469.
49. Lin, K.-H., Hong, S.-T., Wang, H.-T., Lo, Y.-L., Lin, A. M.-Y., Yang, J. C.-H., 2016. Enhancing anticancer effect of gefitinib across the blood-brain barrier model using liposomes modified with one α-helical cell-penetrating peptide or glutathione and tween 80. International Journal of Molecular Sciences 17, 1998.
50. Liu, W., Selomulya, C., Chen, X. D., 2013. Design of polymeric microparticles for pH-responsive and time-sustained drug release. Biochemical Engineering Journal 81, 177-186.
51. Makela, M., Salo, T., Larjava, H., 1998. MMP-9 from TNFα-stimulated keratinocytes binds to cell membranes and type I collagen: a cause for extended matrix degradation in inflammation? Biochemical and Biophysical Research Communications 253, 325-335.
52. Nunes, G. L., de Araújo Etchepare, M., Cichoski, A. J., Zepka, L. Q., Lopes, E. J., Barin, J. S., de Moraes Flores, É. M., dₐ Silva, C. d. B., de Menezes, C. R., 2018. Inulin, hi-maize, and trehalose as thermal protectants for increasing viability of Lactobacillus acidophilus encapsulated by spray drying. LWT 89, 128-133.
53. Palmieri, G. F., Bonacucina, G., Di Martino, P., Martelli, S., 2001. Spray-drying as a method for microparticulate controlled release systems preparation: advantages and limits. I. Water-soluble drugs. Drug Development and Industrial Pharmacy 27, 195-204.
54. Pan, Z., Sun, H., Xie, B., Xia, D., Zhang, X., Yu, D., Li, J., Xu, Y., Wang, Z., Wu, Y., 2018. Therapeutic effects of gefitinib-encapsulated thermosensitive injectable hydrogel in intervertebral disc degeneration. Biomaterials 160, 56-68.
55. Pang, X., Yang, P., Wang, L., Cao, J., Cheng, Y., Sheng, D., Wan, X., Guo, Q., Qian, K., Zhang, Q., 2019. Human serum albumin nanoparticulate system with encapsulation of gefitinib for enhanced anti-tumor effects in non-small cell lung cancer. Journal of Drug Delivery Science and Technology 52, 997-1007.
56. Petersen, P. E., Ogawa, H., 2012. The global burden of periodontal disease: towards integration with chronic disease prevention and control. Periodontology 2000 60, 15-39.
57. Rezvankhah, A., Emam-Djomeh, Z., Askari, G., 2020. Encapsulation and delivery of bioactive compounds using spray and freeze-drying techniques: A review. Drying Technology 38, 235-258.
58. Sedghi, L. M., Bacino, M., Kapila, Y. L., 2021. Periodontal disease: The good, the bad, and the unknown. Frontiers in Cellular and Infection Microbiology, 1210.
59. Siavoshian, S., Segain, J., Kornprobst, M., Bonnet, C., Cherbut, C., Galmiche, J., Blottiere, H., 2000. Butyrate and trichostatin A effects on the proliferation/differentiation of human intestinal epithelial cells: induction of cyclin D3 and p21 expression. Gut 46, 507-514.
60. Soni, M., Malviya, N., Yadav, K. S., 2017. Formulation and evaluation of chitosan based microparticles of gefitinib. International Journal of Pharmacy & Life Sciences 8 (5), 68.
61. Sosa, N., Zamora, M. C., Chirife, J., Schebor, C., 2011. Spray-drying encapsulation of citral in sucrose or trehalose matrices: physicochemical and sensory characteristics. International Journal of Food Science & Technology 46, 2096-2102.
62. Su, W., Jia, N., Li, H., Hao, H., Li, C., 2017. Polymorphism of D-mannitol: Crystal structure and the crystal growth mechanism. Chinese Journal of Chemical Engineering 25, 358-362.
63. Taylor, A. W., 2009. Review of the activation of TGF-beta in immunity. J Leukoc Biol 85, 29-33.
64. Trivanovic, U., Corbin, J. C., Baldelli, A., Peng, W., Yang, J., Kirchen, P., Miller, J. W., Lobo, P., Gagné, S., Rogak, S. N., 2019. Size and morphology of soot produced by a dual-fuel marine engine. Journal of Aerosol Science 138, 105448.
65. Trivanovic, U., Sipkens, T. A., Kazemimanesh, M., Baldelli, A., Jefferson, A. M., Conrad, B. M., Johnson, M. R., Corbin, J. C., Olfert, J. S., Rogak, S. N., 2020. Morphology and size of soot from gas flares as a function of fuel and water addition. Fuel 279, 118478.
66. Trummer, B. J., Iyer, V., Balu-lyer, S. V., O'Connor, R., Straubinger, R. M., 2012. Physicochemical properties of epidermal growth factor receptor inhibitors and development of a nanoliposomal formulation of gefitinib. Journal of Pharmaceutical Sciences 101, 2763-2776.
67. Twarużek, M., Zastempowska, E., Soszczyńska, E., Altyn, I., 2018. The use of in vitro assays for the assessment of cytotoxicity on the example of MTT test. Acta Universitatis Lodziensis. Folia Biologica et Oecologica 14, 23-32.
68. Vicente, J., Pinto, J., Menezes, J., Gaspar, F., 2013. Fundamental analysis of particle formation in spray drying. Powder Technology 247, 1-7.
69. Wang, R., Zhu, J., Dong, X., Shi, M., Lu, C., Springer, T. A., 2012. GARP regulates the bioavailability and activation of TGFβ. Molecular Biology of the Cell 23, 1129-1139.
70. Wang, Z., Wang, H., Vehring, R., 2021. Leucine enhances the dispersibility of trehalose-containing spray-dried powders on exposure to a high-humidity environment. International Journal of Pharmaceutics 601, 120561.
71. Wee, P., Wang, Z., 2017. Epidermal growth factor receptor cell proliferation signaling pathways. Cancers 9, 52.
72. Wong, T., John, P., 2015. Advances in spray drying technology for nanoparticle formation. Springer International Publishing, pp. 329-346.
73. Xu, M., Chen, X., Yin, H., Yin, L., Liu, F., Fu, Y., Yao, J., Deng, X., 2015. Cloning and characterization of the human integrin β6 gene promoter. PLOS One 10, e0121439.
74. Yaffe, H., Buxdorf, K., Shapira, I., Ein-Gedi, S., Moyal-Ben Zvi, M., Fridman, E., Moshelion, M., Levy, M., 2012. LogSpin: a simple, economical and fast method for RNA isolation from infected or healthy plants and other eukaryotic tissues. BMC Research Notes 5, 1-8.
75. Yoshii, H., Neoh, T. L., Furuta, T., Ohkawara, M., 2008. Encapsulation of proteins by spray drying and crystal transformation method. Drying Technology 26, 1308-1312.
76. Yu, X., Yang, G., Shi, Y., Su, C., Liu, M., Feng, B., Zhao, L., 2015. Intracellular targeted co-delivery of shMDR1 and gefitinib with chitosan nanoparticles for overcoming multidrug resistance. International Journal of Nanomedicine 10, 7045.

What is claimed is:

1. A sustained-release pharmaceutical composition for the localized treatment of periodontal disease, the pharmaceutical composition comprising:
a suspension of microparticles, each microparticle comprising:
an epidermal growth factor receptor inhibitor (EGFRI),
a stabilizing sugar, and
a shell comprising a plurality of polymers;
wherein the EGFRI, and the stabilizing sugar are encapsulated in the polymer shell.

2. The sustained-release pharmaceutical composition of claim 1, wherein the plurality of polymers comprises three polymers, a higher molecular weight polymer, an intermediate molecular weight polymer and a lower molecular weight polymer wherein the higher molecular weight polymer has a molecular weight that is greater than a molecular weight of the intermediate molecular weight polymer and the molecular weight of the intermediate molecular weight polymer is greater than a molecular weight of the low molecular weight polymer.

3. The sustained-release pharmaceutical composition of claim 2, wherein the plural polymers are soluble in ethanol and have low solubility in water.

4. The sustained-release pharmaceutical composition of claim 3, wherein the higher molecular weight polymer is poly lactic-co-glycolic acid (PLGA).

5. The sustained-release pharmaceutical composition of claim 4, wherein the intermediate molecular weight polymer is cellulose acetate butyrate (CAB).

6. The sustained-release formulation of claim 5, wherein the low molecular weight polymer is ethyl cellulose (EC).

7. The sustained-release pharmaceutical composition of claim 6, wherein the EGFRI is gefitinib.

8. The sustained-release pharmaceutical composition of claim 7, wherein a weight ratio of the CAB to the PLGA to the EC is about 59:9:24.

9. The sustained-release pharmaceutical composition of claim 7, wherein a ratio of the combined mass of the plural polymers to the mass of the stabilizing sugar in the microparticles is at least 3:1.

10. The sustained-release pharmaceutical composition of claim 9, wherein the stabilizing sugar comprises mannose.

11. The sustained-release pharmaceutical composition of claim 9, wherein the stabilizing sugar comprises trehalose.

12. The sustained-release pharmaceutical composition of claim 9, wherein a projected area equivalent diameter of the microparticles is 20 μm or less.

13. The sustained-release pharmaceutical composition of claim 12, wherein the projected area equivalent diameter of the microparticles is in the range of about 3 μm to about 10 μm.

14. The sustained-release pharmaceutical composition of claim 7 wherein the microparticles are 3% to 7% gefitinib by weight.

15. The sustained-release pharmaceutical composition of claim 14 wherein, when the pharmaceutical composition is mixed with a liquid selected from artificial saliva and cell culture medium, a release rate of the gefitinib from the microparticles is sufficiently slow that no more than 95% of the gefitinib has been released into the liquid in a period of 350 hours.

16. The sustained-release pharmaceutical composition of claim 7 comprising a gel, wherein the microparticles are suspended in the gel.

17. The sustained-release pharmaceutical composition of claim 1, wherein the shell comprises an outermost layer, an intermediate layer inwardly adjacent to the outermost layer and an inner layer inwardly adjacent to the intermediate layer.

18. The sustained-release pharmaceutical composition of claim 17, wherein the outermost layer comprises PLGA, the intermediate layer comprises CAB and the inner layer comprises EC.

19. The sustained-release pharmaceutical composition of claim 1 wherein the microparticles have a surface morphology that is folded and crumpled.

20. A dosage regime for treating periodontal disease (PD), the dosage regime consisting of administering into a periodontal pocket of a patient suffering from PD a dose unit of the sustained release pharmaceutical composition according to claim 1, wherein the EGFRI comprises gefitinib, and the dose unit consists essentially of an amount of the suspension such that the microparticles of the dose unit contain a total weight of gefitinib in the range of 26 ng to 1 μg.

21. A method of treating periodontal disease (PD), comprising: administering a dose unit of the sustained release pharmaceutical composition according to claim 1 into a periodontal pocket of a subject suffering from PD, wherein the dose unit contains a total weight of gefitinib in the range of 26 ng to 1 μg.

22. The method according to claim 21 comprising repeating administration of the dose unit to the subject on an administration schedule of 10 to 15 weeks until symptoms of PD are relieved.
